SEQUENCE LISTING (36 sequences)

SEQ ID NO: 1 - protein, 314 aa, Mycoplasma hyopneumoniae
KPIKIALIGAGNVGNSFLYAAMNQGLASEYGIIDINPDFADGNAFDFEDASASLPFPISVSRYEYKDLKDADFIVITAGRPQKPGETRLELVADNIRIIREIALKVKESGFSGISIIVANPVDIITRAYRDASGFSDQKVIGSGTVLDTARLQFAIAKRAKVSPNSVQAYVMGEHGDSSFVAYSNIKIAGEYFCAYSKLTGIDSSNYEKELEYPVSRRAYEIINRKRATFYGIGAAIAKIVSNIIKDTKNIMIAGANLRGEYGFHGVNIGVPVVLGANGIEKIIEISLNDKEKEKFAKSVAIIDKIYQDAIKNI

SEQ ID NO: 2 - protein, 368 aa, Mycoplasma hyopneumoniae
KHKVSNDSIRIALTDPDNPRWISAQKDIISYVDETEAATSTITKNQDAQNNWLTQQANLSPAPKGFIIAPENGSGVGTAVNTIADKGIPIVAYDRLITGSDKYDWYVSFDNEKVGELQGLSLAAGLLGKEDGAFDSIDQMNEYLKSHMPQETISFYTIAGSQDDNNSQYFYNGAMKVLKELMKNSQNKIIDLSPEGENAVYVPGWNYGTAGQRIQSFLTINKDPAGGNKIKAVGSKPASIFKGFLAPNDGMAEQAITKLKLEGFDTQKIFVTGQDYNDKAKTFIKDGDQNMTIYKPDKVLGKVAVEVLRVLIAKKNKASRSEVENELKAKLPNISFKYDNQTYKVQGKNINTILVSPVIVTKANVDNP

SEQ ID NO: 3 - protein, 245 aa, Mycoplasma hyopneumoniae
GEISDVLLLDVTPLTLGIETLGGIATPLIPRNTTIPVTKSQIFSTAEDNQTEVTISVVQGERQLAADNKMLGRFNLSGIEAAPRGLPQIEVSFSIDVNGITTVSAKDKKTGKEQTITIKNTSTLSEEEINKMIQEAEENREADALKKDKIETTVRAEGLINQLEKSITDQGEKIDPKQKELLEKQIQELKDLLKEDKTDELKLKLDQIEAAAQSFAQATAQQANTSESDPKADDSNTIDAEIKQD

SEQ ID NO: 4 - protein, 106 aa, Mycoplasma hyopneumoniae
KQDEIKKFTFDLLYKLIELEKDYLYDLYSEVGLAESAIKFSIYNAGKFLQNLGYDSPFSKEETEIEPEIFSQLSARADENHDFFSGNGSSYVMALAEETEDEDWEF

SEQ ID NO: 5 - protein, 144 aa, Mycoplasma hyopneumoniae
AAKPEAAKPVAAKPETTKPVAAKPEAAKPEAAKPVAAKPEAAKPVAAKPEAAKPVAAKPEAAKPVAAKPEAAKPVATNTGFSLTNKPKEDYFPMTEGSSNQGKKSEGTPNQGKKAEGAPNQGKKAEGTPNQGKKAEGAPSQQSP

SEQ ID NO: 6 - protein, 162 aa, Mycoplasma hyopneumoniae
SEFKEWRLDESAFSSLTFPKSQKSEGTQNQGKKAEGAPNQSKKSEENSNKLTEYIQELGTKVEKSLKSKGKNYSAEVEKIIEAFSGGYKFLDFALVEQTPKPETPKTEAAKPETTKPVAARPEAAKVAAKPSAAKPVSSPAPKKSTLYVRVLIRKKENKQVK

SEQ ID NO: 7 - protein, 25 aa, Mycoplasma hyopneumoniae
QKKAVRKEEELETYNPKDEFNILNP

SEQ ID NO: 8 - protein, 24 aa, Mycoplasma hyopneumoniae
VNNDPNYKIEDLKVIKNEAGDHQL

SEQ ID NO: 9 - protein, 261 aa, Pyrococcus abyssi
KRIFVQNRELVVPGTLLAQGPYKNGRGTFREGSRIYSTVIGLVDIKGNTIRVIPLEGPYIPEVGDNVIGKIVDVKFSSWVVDIGAPYPANLKIQDFTDEKIDLLRTDLRKFFDIGDIIYAKVKAITEVNNIDLTTKGMPFNGGPLKGGQIVKITPSRVPRVIGRGGSMINMIKKLTMTRIIVGQNGWIWVSGKNDALEKLAIEAILKIDKESHTRGLTDRIKALLLSRLQELKEKGVIEEIPKLEEEPQGEDEVNGNDGEA

SEQ ID NO: 10 - protein, 244 aa, Pyrococcus abyssi
EKPEGLKLIDENGRRIDGRKKYELRPIKMEVGVLKNANGSAYIEWGKNKIIAAVYGPRELHPKHLQRPDRAILRVRYNMAPFSVEERKKPGPDRRSIEISKVIKGALEPALILEMFPRTAIDVFIEVLQADAGTRVAGITAASLALADAGIPMRDLVAACAAGKIEGEIVLDLNKEEDNYGEADVPVAIMPLKNDITLLQMDGYLTKDEFIEAVKLAIKGAKAVYQKQREALKEKYLKIAQEVE

SEQ ID NO: 11 - protein, 272 aa, Pyrococcus abyssi
SDNEIVAGIMRDHIINLLKEGKRIDDRGFEDYRPIEIEVGVIEKAEGSALVKLGSTQVLVGIKTSLGEPFPDTPNMGVMTTNVELVPLASPTFEPGPPDERAIELARVIDRGIRESKALNLEKMVIVPGKIVRVVFIDVHVLDHDGNLMDAIGIAAIAALLNARVPKVRYNEETGEVETLDETEPLPVEKIPVPVTFAKIGNILVVDPSLDEELVMDGKITITTDETGHISAVQKSEGGAFKLEEVMYAVETAFKKAEEIRKLILEAVEKAK

SEQ ID NO: 12 - DNA, 942 bp, Artificial Sequence
  Gene sintetico para o antigeno P36
aaaccgattaagattgctctgattggtgcaggcaatgtaggcaactctttcctgtacgcggcaatgaatcagggtctggcgtctgaatatggtattattgatattaacccggatttcgctgacggtaatgcattcgatttcgaggacgcttccgcttcccttccgttcccaatttccgtttcccgttatgaatacaaagacctgaaagatgcagactttattgttattaccgcgggccgaccgcagaagccgggtgagacccgcctggaactggttgctgacaacattcgcatcatccgcgaaattgccctgaaagtgaaagaaagcggtttctctggtatttctattattgtagctaacccggtagatattatcacccgtgcttatcgcgatgcttccggtttctccgaccaaaaagttatcggttccggcaccgtgctggacaccgcgcgtctgcaattcgccattgcaaaacgcgcgaaagttagcccgaactcggttcaagcatacgttatgggtgagcacggtgactctagctttgtagcctacagcaacattaagattgcgggtgaatatttctctgcgtacagcaaactgaccggtattgacagctctaactacgaaaaagaactggaatacccggtgtcccgtcgagcgtacgaaattattaaccgaaaacgtgcgactttctatggtatcggcgctgcaattgcaaagattgtttctaacatcattaaagacaccaagaacattatgattgccggcgcgaacctgcgcggtgaatatggtttccacggtgtaaacattggtgtgccggttgtactgggtgcaaacggcatcgagaaaattatcgaaatttctctgaacgataaagaaaaagaaaagttcgcaaaatccgtagcgattattgataaaatctatcaggatgcgattaaaaacatt

SEQ ID NO: 13 - DNA, 1104 bp, Artificial Sequence
  Gene sintetico para o antigeno P46
aaacacaaagtatctaacgactccattcgcattgcactgactgaccctgataacccgcgctggatttctgctcagaaagatattatttcctatgttgatgaaaccgaggcggctacttctaccattaccaaaaaccaggatgcccagaacaactggctgacccagcaggctaacctgtccccggcaccgaaaggctttattatcgctccggaaaacggttctggtgtgggtactgccgttaacactattgctgacaaaggtatccctattgtagcttatgaccgcctgattactggtagcgacaaatatgactggtacgtgtctttcgataacgaaaaggttggcgaactgcaaggcctgtctctggcggcgggtctgctgggtaaagaggacggtgcttttgactctattgaccagatgaacgagtacctgaaatctcacatgccgcaggaaactatcagcttctacaccattgccggcagccaggacgataacaacagccaatacttctacaacggtgccatgaaagtactgaaagagctgatgaaaaacagccagaacaaaattattgacctgtctccggaaggcgaaaacgcggtttacgttccgggttggaactacggtactgccggtcaacgcattcagtctttcctgactattaacaaggacccggccggtggtaataaaattaaggctgtaggttccaagccggcgtctatctttaaaggtttcctggcaccgaatgacggcatggcagagcaggcaattaccaagctgaaactggaaggcttcgatacccagaaaattttcgtaaccggtcaggattataacgacaaagcgaaaacttttattaaagacggcgaccagaacatgaccatttataaaccggacaaagttctgggtaaagtggcagtggaagttctgcgtgttctgattgcgaaaaaaaacaaagccagccgaagcgaagttgaaaacgagctgaaagcgaaactgccgaacatctccttcaagtatgataaccagacctacaaggtgcaaggcaagaacattaatactatcctggtttctccggtgattgtaaccaaagcgaatgtggacaaccct

SEQ ID NO: 14 - DNA, 735 bp, Artificial Sequence
  Gene sintetico para o antigeno HSP
ggtgaaatttctgacgtgctgctgctggacgtaaccccgctgaccctgggcattgagactctgggtggcattgctaccccgctgattccgcgaaacaccaccattccagtgaccaaaagccaaattttctccaccgctgaagataaccagaccgaagttaccatctccgttgtgcaaggtgaacgacagctggcagccgacaacaagatgctgggtcgctttaacctgagcggcatcgaagcggctccgcgtggtctgccacaaattgaagtgtccttcagcatcgatgtaaacggcatcaccactgtttccgctaaagacaaaaaaaccggtaaagaacagaccattaccatcaagaatacctccaccctgtctgaagaagaaattaacaaaatgattcaggaagctgaggaaaaccgtgaagccgatgcactgaaaaaagacaaaattgaaaccaccgtacgtgcggaaggcctgattaaccagctggaaaaatctattactgaccagggtgagaaaattgacccgaaacagaaagaactgctggaaaagcagattcaggaactgaaagacctgctgaaagaggataaaaccgacgaactgaaactgaaactggaccagattgaagctgcggcccaatccttcgcgcaggcaaccgcgcagcaagcgaacaccagcgaatctgacccgaaagcggacgatagcaacaccatcgacgcggagattaaacaagat

SEQ ID NO: 15 - DNA, 318 bp, Artificial Sequence
  Gene sintetico para o antigeno NrdF
aaacaggatgaaatcaaaaaattcaccttcgacctgctgtacaaactgattgaactggaaaaagattacctgtacgacctgtactccgaagtgggcctggcggaaagcgccatcaagttcagcatctacaacgctggcaaattcctgcaaaacctgggctacgattctccgttttctaaagaagagaccgaaatcgagccggagattttctcccagctgagcgctcgcgcagatgagaaccatgacttcttctctggtaacggcagcagctacgtgatggcgctggcagaggaaaccgaagacgaagattgggagttc

SEQ ID NO: 16 - DNA, 441 bp, Artificial Sequence
  Gene sintetico para o antigeno P97R1R2
gcggcgaaaccggaggcagcgaaaccggttgcggctaaaccggagaccaccaaaccggtggctgctaaaccggaagcggccaaaccggaagctgcgaaaccggtggcggcgaagccggaggctgccaaaccggtagctgctaagccggaagcggctaagccggtggcagcaaaaccggaggcggcaaaaccggtggccgccaagccggaagctgccaagccggttgcaaccaacactggtttttctctgaccaacaaaccgaaagaagactactttccgatgaccgaaggctccagcaaccagggtaaaaagtccgaaggtactccgaaccagggtaagaaagcggagggtgcaccgaaccagggcaaaaaagcggaaggcaccccgaaccaaggtaaaaaagcagagggtgcgccgagccagcagagcccgaccaccgaa

SEQ ID NO: 17 - DNA, 393 bp, Artificial Sequence
  Gene sintetico para o antigeno MHP271
ccgaaatcccaaaaatctgaaggtactcaaaaccagggtaaaaaagctgaaggtgccccgaatcagtccaaaaaaagcgaggaaaactccaacaaactgactgaatacattcaagaactgggcactaaagtggagaagagcctgaaatctaaaggtaaaaactacagcgcagaagttgagaagattattgaagcattcagcggcggctataagttcctggatttcgcgctggttgaacagaccccgaaaccggaaaccccgaaaactgaagctgctaaaccggaaactactaaaccggtggcggcgcgcccggaagctgcgaaagttgctgcgaaaccgtccgctgcgaaacctgtttccagcccggcgccgaaaaaatctaccctgtacgtg

SEQ ID NO: 18 - DNA, 78 bp, Artificial Sequence
  Gene sintetico para o antigeno P216-A
ggccagaagaaggcggtacgtaaggaagaggaactggaaacctacaacccgaaagacgagtttaatatcctgaacccg

SEQ ID NO: 19 - DNA, 72 bp, Artificial Sequence
  Gene sintetico para o antigeno P216-A
gtgaacaacgacccgaactacaaaattgaagacctgaaggtgattaagaacgaggcaggtgaccatcagctt

SEQ ID NO: 20 - DNA, 783 bp, Artificial Sequence
  Gene para proteina Rrp4 do exossomo de P. abyssi
aaacgtatcttcgtacagaatcgtgaactggtggtgccgggtactctgctggcccagggtccttataaaaacggtcgtggcactttccgtgaaggtagccgtatctactctaccgttatcggtctggtggacatcaaaggcaacaccattcgtgtgattcctctggagggtccgtacatcccggaagtaggtgacaacgttatcggtaaaattgtggacgttaagttctctagctgggttgtagacattggtgcgccgtatccggccaacttgaagattcaggacttcactgatgaaaaaattgatttgctgcgtactgaccttcgtaagttctttgatattggtgacattatctacgctaaagtaaaagccatcaccgaagttaacaacattgacctgacgaccaaaggcatgccgtttaacggtggtccgctgaaaggtggtcaaatcgtgaaaattacgccgtctcgtgtgccgcgtgttatcggtcgcggtggcagcatgattaacatgattaaaaaactgaccatgactcgtattattgttggccaaaacggctggatttgggtgtctggcaaaaacgatgcgctggaaaaacttgctattgaggccattctgaaaattgacaaagagtctcatacccgtggtctgactgaccgtattaaagcgctgctgctgtcccgcctgcaagaactgaaagaaaaaggtgttattgaagagattccgaaactggaagaagagccgcagggcgaagatgaagtaaacggcaatgacggtgaagct

SEQ ID NO: 21 - DNA, 732 bp, Artificial Sequence
  Gene para proteina Rrp41 do exossomo de P. abyssi
gagaaaccggaaggtctgaaactgattgatgaaaacggccgccgtattgacggtcgtaaaaaatacgaactgcgtccgattaaaatggaagtgggtgtactgaaaaacgctaacggttccgcgtacatcgaatggggcaaaaacaaaattatcgcagcggtttacggtccgcgtgaactccacccgaaacaccttcagcgtccggaccgcgccatcctgcgtgtacgttacaacatggccccgttctctgttgaagagactaagagcccgggtccggaccgtcgtagcattgagatttctaaagtaattaaaggtgcgctggaaccggcacttattctggaaatgttcccgcgtactgctattgatgtattcatcgaagttctgcaagcggatgccggtacgcgcgttgcaggtatcaccgcggctagcctggcactggccgacgctggtattccgatgcgcgacctggttgccgcttgtgcggcgggtaaaatcgaaggcgaaattgttctggacctgaacaaagaagaagataactacggcgaagcggacgtgccggtagctatcatgccgctgaagaacgatattaccctgctgcaaatggatggttacctgaccaaggacgagttcatcgaggcggttaaactggctattaaaggtgctaaggcggtgtatcagaaacagcgtgaagcactgaaagaaaagtatctgaaaattgcccaggaagttgag

SEQ ID NO: 22 - DNA, 819 bp, Artificial Sequence
  Gene para proteina Rrp42 do exossomo de P. abyssi
tctgataatgaaatcgtggcgggtattatgcgcgaccatattattaacctgcttaaggaaggtaaacgtattgacgaccgcggtttcgaagattaccgcccgattgaaattgaggtaggtgttattgaaaaagcggagggtagcgcgctggtgaaactgggtagcactcaggttctggtgggtattaaaacctctctgggcgagccgtttccggacaccccgaacatgggcgtgatgaccaccaatgttgaacttgttccgctggcgtctccgactttcgaaccgggcccgccggacgagcgtgctatcgaactggcacgtgttattgaccgtggtattcgtgagtccaaagcactgaatctggaaaaaatggttatcgtgccgggcaaaattgttcgcgtagtttttatcgacgttcacgttcttgaccacgacggtaacctgatggacgccatcggtattgcagctatcgccgcactgctgaacgcacgtgttccgaaagttcgctacaacgaagaaacgggtgaagttgaaaccctggatgaaactgaaccgctgccggttgaaaagattccggttccggttactttcgctaaaattggtaatatcctggtagttgacccgagcctggatgaagagctcgtgatggacggcaaaattaccattaccactgacgaaaccggccatatttctgccgttcagaaatctgaaggtggcgctttcaaactggaagaagtaatgtacgcggtggaaactgcattcaagaaggcggaagaaatccgtaaactgattctggaagctgttgagaaggccaaacag

SEQ ID NO: 23 - DNA, 145 bp, Artificial Sequence
  Promotor para o gene de Rrp41
taactgcaggcttaagtcgaacagaaagtaatcgtattgtacacggccgcaacttcgaaattaatacgactcactatagggagaccacaacggtttcccatcttagtatattagttaagtataagaaggagatataaacatggca

SEQ ID NO: 24 - DNA, 144 bp, Artificial Sequence
  Promotor para o gene de Rrp42
taagaattcgcttaagtcgaacagaaagtaatcgtattgtacacggccgcataatcgaaattaatacgactcactatagggagaccacaacggtttcccatcttagtatattagttaagtataagaaggagatataccatgggt

SEQ ID NO: 25 - DNA, 6764 bp, Artificial Sequence
  Gene para o complexo EXOMYC
  CDS (4)..(2217): Poliproteina 1 Rrp4-P36-MHP271
  CDS (2354)..(4651): Poliproteina 2 Rrp41-P46-P97R1R2
  CDS (4787)..(6760): Poliproteina 3 Rrp42-HSP-NrdF
catatggccaaacgtatcttcgtacagaatcgtgaactggtggtgccgggtactctgctggcccagggtccttataaaaacggtcgtggcactttccgtgaaggtagccgtatctactctaccgttatcggtctggtggacatcaaaggcaacaccattcgtgtgattcctctggagggtccgtacatcccggaagtaggtgacaacgttatcggtaaaattgtggacgttaagttctctagctgggttgtagacattggtgcgccgtatccggccaacttgaagattcaggacttcactgatgaaaaaattgatttgctgcgtactgaccttcgtaagttctttgatattggtgacattatctacgctaaagtaaaagccatcaccgaagttaacaacattgacctgacgaccaaaggcatgccgtttaacggtggtccgctgaaaggtggtcaaatcgtgaaaattacgccgtctcgtgtgccgcgtgttatcggtcgcggtggcagcatgattaacatgattaaaaaactgaccatgactcgtattattgttggccaaaacggctggatttgggtgtctggcaaaaacgatgcgctggaaaaacttgctattgaggccattctgaaaattgacaaagagtctcatacccgtggtctgactgaccgtattaaagcgctgctgctgtcccgcctgcaagaactgaaagaaaaaggtgttattgaagagattccgaaactggaagaagagccgcagggcgaagatgaagtaaacggcaatgacggtgaagctagatctggtgtgaacaacgacccgaactacaaaattgaagacctgaaggtgattaagaacgaggcaggtgaccatcagcttggctccaaaccgattaagattgctctgattggtgcaggcaatgtaggcaactctttcctgtacgcggcaatgaatcagggtctggcgtctgaatatggtattattgatattaacccggatttcgctgacggtaatgcattcgatttcgaggacgcttccgcttcccttccgttcccaatttccgtttcccgttatgaatacaaagacctgaaagatgcagactttattgttattaccgcgggccgaccgcagaagccgggtgagacccgcctggaactggttgctgacaacattcgcatcatccgcgaaattgccctgaaagtgaaagaaagcggtttctctggtatttctattattgtagctaacccggtagatattatcacccgtgcttatcgcgatgcttccggtttctccgaccaaaaagttatcggttccggcaccgtgctggacaccgcgcgtctgcaattcgccattgcaaaacgcgcgaaagttagcccgaactcggttcaagcatacgttatgggtgagcacggtgactctagctttgtagcctacagcaacattaagattgcgggtgaatatttctctgcgtacagcaaactgaccggtattgacagctctaactacgaaaaagaactggaatacccggtgtcccgtcgagcgtacgaaattattaaccgaaaacgtgcgactttctatggtatcggcgctgcaattgcaaagattgtttctaacatcattaaagacaccaagaacattatgattgccggcgcgaacctgcgcggtgaatatggtttccacggtgtaaacattggtgtgccggttgtactgggtgcaaacggcatcgagaaaattatcgaaatttctctgaacgataaagaaaaagaaaagttcgcaaaatccgtagcgattattgataaaatctatcaggatgcgattaaaaacattccgaaatcccaaaaatctgaaggtactcaaaaccagggtaaaaaagctgaaggtgccccgaatcagtccaaaaaaagcgaggaaaactccaacaaactgactgaatacattcaagaactgggcactaaagtggagaagagcctgaaatctaaaggtaaaaactacagcgcagaagttgagaagattattgaagcattcagcggcggctataagttcctggatttcgcgctggttgaacagaccccgaaaccggaaaccccgaaaactgaagctgctaaaccggaaactactaaaccggtggcggcgcgcccggaagctgcgaaagttgctgcgaaaccgtccgctgcgaaacctgtttccagcccggcgccgaaaaaatctaccctgtacgtgtaactgcaggcttaagtcgaacagaaagtaatcgtattgtacacggccgcaacttcgaaattaatacgactcactatagggagaccacaacggtttcccatcttagtatattagttaagtataagaaggagatataaacatggcagagaaaccggaaggtctgaaactgattgatgaaaacggccgccgtattgacggtcgtaaaaaatacgaactgcgtccgattaaaatggaagtgggtgtactgaaaaacgctaacggttccgcgtacatcgaatggggcaaaaacaaaattatcgcagcggtttacggtccgcgtgaactccacccgaaacaccttcagcgtccggaccgcgccatcctgcgtgtacgttacaacatggccccgttctctgttgaagagactaagagcccgggtccggaccgtcgtagcattgagatttctaaagtaattaaaggtgcgctggaaccggcacttattctggaaatgttcccgcgtactgctattgatgtattcatcgaagttctgcaagcggatgccggtacgcgcgttgcaggtatcaccgcggctagcctggcactggccgacgctggtattccgatgcgcgacctggttgccgcttgtgcggcgggtaaaatcgaaggcgaaattgttctggacctgaacaaagaagaagataactacggcgaagcggacgtgccggtagctatcatgccgctgaagaacgatattaccctgctgcaaatggatggttacctgaccaaggacgagttcatcgaggcggttaaactggctattaaaggtgctaaggcggtgtatcagaaacagcgtgaagcactgaaagaaaagtatctgaaaattgcccaggaagttgagggatcccctaaacacaaagtatctaacgactccattcgcattgcactgactgaccctgataacccgcgctggatttctgctcagaaagatattatttcctatgttgatgaaaccgaggcggctacttctaccattaccaaaaaccaggatgcccagaacaactggctgacccagcaggctaacctgtccccggcaccgaaaggctttattatcgctccggaaaacggttctggtgtgggtactgccgttaacactattgctgacaaaggtatccctattgtagcttatgaccgcctgattactggtagcgacaaatatgactggtacgtgtctttcgataacgaaaaggttggcgaactgcaaggcctgtctctggcggcgggtctgctgggtaaagaggacggtgcttttgactctattgaccagatgaacgagtacctgaaatctcacatgccgcaggaaactatcagcttctacaccattgccggcagccaggacgataacaacagccaatacttctacaacggtgccatgaaagtactgaaagagctgatgaaaaacagccagaacaaaattattgacctgtctccggaaggcgaaaacgcggtttacgttccgggttggaactacggtactgccggtcaacgcattcagtctttcctgactattaacaaggacccggccggtggtaataaaattaaggctgtaggttccaagccggcgtctatctttaaaggtttcctggcaccgaatgacggcatggcagagcaggcaattaccaagctgaaactggaaggcttcgatacccagaaaattttcgtaaccggtcaggattataacgacaaagcgaaaacttttattaaagacggcgaccagaacatgaccatttataaaccggacaaagttctgggtaaagtggcagtggaagttctgcgtgttctgattgcgaaaaaaaacaaagccagccgaagcgaagttgaaaacgagctgaaagcgaaactgccgaacatctccttcaagtatgataaccagacctacaaggtgcaaggcaagaacattaatactatcctggtttctccggtgattgtaaccaaagcgaatgtggacaaccctggggcggcgaaaccggaggcagcgaaaccggttgcggctaaaccggagaccaccaaaccggtggctgctaaaccggaagcggccaaaccggaagctgcgaaaccggtggcggcgaagccggaggctgccaaaccggtagctgctaagccggaagcggctaagccggtggcagcaaaaccggaggcggcaaaaccggtggccgccaagccggaagctgccaagccggttgcaaccaacactggtttttctctgaccaacaaaccgaaagaagactactttccgatgaccgaaggctccagcaaccagggtaaaaagtccgaaggtactccgaaccagggtaagaaagcggagggtgcaccgaaccagggcaaaaaagcggaaggcaccccgaaccaaggtaaaaaagcagagggtgcgccgagccagcagagcccgaccaccgaataagaattcgcttaagtcgaacagaaagtaatcgtattgtacacggccgcataatcgaaattaatacgactcactatagggagaccacaacggtttcccatcttagtatattagttaagtataagaaggagatataccatgggttctgataatgaaatcgtggcgggtattatgcgcgaccatattattaacctgcttaaggaaggtaaacgtattgacgaccgcggtttcgaagattaccgcccgattgaaattgaggtaggtgttattgaaaaagcggagggtagcgcgctggtgaaactgggtagcactcaggttctggtgggtattaaaacctctctgggcgagccgtttccggacaccccgaacatgggcgtgatgaccaccaatgttgaacttgttccgctggcgtctccgactttcgaaccgggcccgccggacgagcgtgctatcgaactggcacgtgttattgaccgtggtattcgtgagtccaaagcactgaatctggaaaaaatggttatcgtgccgggcaaaattgttcgcgtagtttttatcgacgttcacgttcttgaccacgacggtaacctgatggacgccatcggtattgcagctatcgccgcactgctgaacgcacgtgttccgaaagttcgctacaacgaagaaacgggtgaagttgaaaccctggatgaaactgaaccgctgccggttgaaaagattccggttccggttactttcgctaaaattggtaatatcctggtagttgacccgagcctggatgaagagctcgtgatggacggcaaaattaccattaccactgacgaaaccggccatatttctgccgttcagaaatctgaaggtggcgctttcaaactggaagaagtaatgtacgcggtggaaactgcattcaagaaggcggaagaaatccgtaaactgattctggaagctgttgagaaggccaaacagctcgagggccagaagaaggcggtacgtaaggaagaggaactggaaacctacaacccgaaagacgagtttaatatcctgaacccgggtgaaatttctgacgtgctgctgctggacgtaaccccgctgaccctgggcattgagactctgggtggcattgctaccccgctgattccgcgaaacaccaccattccagtgaccaaaagccaaattttctccaccgctgaagataaccagaccgaagttaccatctccgttgtgcaaggtgaacgacagctggcagccgacaacaagatgctgggtcgctttaacctgagcggcatcgaagcggctccgcgtggtctgccacaaattgaagtgtccttcagcatcgatgtaaacggcatcaccactgtttccgctaaagacaaaaaaaccggtaaagaacagaccattaccatcaagaatacctccaccctgtctgaagaagaaattaacaaaatgattcaggaagctgaggaaaaccgtgaagccgatgcactgaaaaaagacaaaattgaaaccaccgtacgtgcggaaggcctgattaaccagctggaaaaatctattactgaccagggtgagaaaattgacccgaaacagaaagaactgctggaaaagcagattcaggaactgaaagacctgctgaaagaggataaaaccgacgaactgaaactgaaactggaccagattgaagctgcggcccaatccttcgcgcaggcaaccgcgcagcaagcgaacaccagcgaatctgacccgaaagcggacgatagcaacaccatcgacgcggagattaaacaagatggttccgggaaacaggatgaaatcaaaaaattcaccttcgacctgctgtacaaactgattgaactggaaaaagattacctgtacgacctgtactccgaagtgggcctggcggaaagcgccatcaagttcagcatctacaacgctggcaaattcctgcaaaacctgggctacgattctccgttttctaaagaagagaccgaaatcgagccggagattttctcccagctgagcgctcgcgcagatgagaaccatgacttcttctctggtaacggcagcagctacgtgatggcgctggcagaggaaaccgaagacgaagattgggagttctaagctt

SEQ ID NO: 26 - protein, 737 aa, Artificial Sequence
  Synthetic Construct
MAKRIFVQNRELVVPGTLLAQGPYKNGRGTFREGSRIYSTVIGLVDIKGNTIRVIPLEGPYIPEVGDNVIGKIVDVKFSSWVVDIGAPYPANLKIQDFTDEKIDLLRTDLRKFFDIGDIIYAKVKAITEVNNIDLTTKGMPFNGGPLKGGQIVKITPSRVPRVIGRGGSMINMIKKLTMTRIIVGQNGWIWVSGKNDALEKLAIEAILKIDKESHTRGLTDRIKALLLSRLQELKEKGVIEEIPKLEEEPQGEDEVNGNDGEARSGVNNDPNYKIEDLKVIKNEAGDHQLGSKPIKIALIGAGNVGNSFLYAAMNQGLASEYGIIDINPDFADGNAFDFEDASASLPFPISVSRYEYKDLKDADFIVITAGRPQKPGETRLELVADNIRIIREIALKVKESGFSGISIIVANPVDIITRAYRDASGFSDQKVIGSGTVLDTARLQFAIAKRAKVSPNSVQAYVMGEHGDSSFVAYSNIKIAGEYFSAYSKLTGIDSSNYEKELEYPVSRRAYEIINRKRATFYGIGAAIAKIVSNIIKDTKNIMIAGANLRGEYGFHGVNIGVPVVLGANGIEKIIEISLNDKEKEKFAKSVAIIDKIYQDAIKNIPKSQKSEGTQNQGKKAEGAPNQSKKSEENSNKLTEYIQELGTKVEKSLKSKGKNYSAEVEKIIEAFSGGYKFLDFALVEQTPKPETPKTEAAKPETTKPVAARPEAAKVAAKPSAAKPVSSPAPKKSTLYV

SEQ ID NO: 27 - protein, 765 aa, Artificial Sequence
  Synthetic Construct
MAEKPEGLKLIDENGRRIDGRKKYELRPIKMEVGVLKNANGSAYIEWGKNKIIAAVYGPRELHPKHLQRPDRAILRVRYNMAPFSVEETKSPGPDRRSIEISKVIKGALEPALILEMFPRTAIDVFIEVLQADAGTRVAGITAASLALADAGIPMRDLVAACAAGKIEGEIVLDLNKEEDNYGEADVPVAIMPLKNDITLLQMDGYLTKDEFIEAVKLAIKGAKAVYQKQREALKEKYLKIAQEVEGSPKHKVSNDSIRIALTDPDNPRWISAQKDIISYVDETEAATSTITKNQDAQNNWLTQQANLSPAPKGFIIAPENGSGVGTAVNTIADKGIPIVAYDRLITGSDKYDWYVSFDNEKVGELQGLSLAAGLLGKEDGAFDSIDQMNEYLKSHMPQETISFYTIAGSQDDNNSQYFYNGAMKVLKELMKNSQNKIIDLSPEGENAVYVPGWNYGTAGQRIQSFLTINKDPAGGNKIKAVGSKPASIFKGFLAPNDGMAEQAITKLKLEGFDTQKIFVTGQDYNDKAKTFIKDGDQNMTIYKPDKVLGKVAVEVLRVLIAKKNKASRSEVENELKAKLPNISFKYDNQTYKVQGKNINTILVSPVIVTKANVDNPGAAKPEAAKPVAAKPETTKPVAAKPEAAKPEAAKPVAAKPEAAKPVAAKPEAAKPVAAKPEAAKPVAAKPEAAKPVATNTGFSLTNKPKEDYFPMTEGSSNQGKKSEGTPNQGKKAEGAPNQGKKAEGTPNQGKKAEGAPSQQSPTTE

SEQ ID NO: 28 - protein, 657 aa, Artificial Sequence
  Synthetic Construct
MGSDNEIVAGIMRDHIINLLKEGKRIDDRGFEDYRPIEIEVGVIEKAEGSALVKLGSTQVLVGIKTSLGEPFPDTPNMGVMTTNVELVPLASPTFEPGPPDERAIELARVIDRGIRESKALNLEKMVIVPGKIVRVVFIDVHVLDHDGNLMDAIGIAAIAALLNARVPKVRYNEETGEVETLDETEPLPVEKIPVPVTFAKIGNILVVDPSLDEELVMDGKITITTDETGHISAVQKSEGGAFKLEEVMYAVETAFKKAEEIRKLILEAVEKAKQLEGQKKAVRKEEELETYNPKDEFNILNPGEISDVLLLDVTPLTLGIETLGGIATPLIPRNTTIPVTKSQIFSTAEDNQTEVTISVVQGERQLAADNKMLGRFNLSGIEAAPRGLPQIEVSFSIDVNGITTVSAKDKKTGKEQTITIKNTSTLSEEEINKMIQEAEENREADALKKDKIETTVRAEGLINQLEKSITDQGEKIDPKQKELLEKQIQELKDLLKEDKTDELKLKLDQIEAAAQSFAQATAQQANTSESDPKADDSNTIDAEIKQDGSGKQDEIKKFTFDLLYKLIELEKDYLYDLYSEVGLAESAIKFSIYNAGKFLQNLGYDSPFSKEETEIEPEIFSQLSARADENHDFFSGNGSSYVMALAEETEDEDWEF

SEQ ID NO: 29 - DNA, 23 bp, Artificial Sequence
  Primer inverso para sequenciamento de insertos em vetor T7
tgtggtctccctatagtgagtcg

SEQ ID NO: 30 - DNA, 32 bp, Artificial Sequence
  Primer inverso para amplificacao do antigeno P36
ggctgcagttaaatgtttttaatcgcatcctg

SEQ ID NO: 31 - DNA, 27 bp, Artificial Sequence
  Primer forward para amplificacao do antigeno MHP271
ccagatctccgaaatcccaaaaatctg

SEQ ID NO: 32 - DNA, 25 bp, Artificial Sequence
  Primer forward para amplificacao da proteina Rrp41
agcatatggcagagaaaccggaagg

SEQ ID NO: 33 - DNA, 34 bp, Artificial Sequence
  Primer inverso para amplificacao do antigeno P46
ccgaattcttaagggttgtccacattcgctttgg

SEQ ID NO: 34 - DNA, 31 bp, Artificial Sequence
  Primer forward para amplificacao do antigeno P97R1R2
aaggatccgcgaaaccggaggcagcgaaacc

SEQ ID NO: 35 - DNA, 31 bp, Artificial Sequence
  Primer inverso para amplificacao do antigeno HSP
ccaagcttaatcttgtttaatctccgcgtcg

SEQ ID NO: 36 - DNA, 37 bp, Artificial Sequence
  Primer forward para amplificacao do antigeno NrdF
ggctcgaggggaaacaggatgaaatcaaaaaattcac

(19) United States
(12) Patent Application Publication
(10) Pub. No.: US 2017/0274062 A1
(43) Pub. Date: Sep. 28, 2017

(54) COMPLEX OF IMMUNOGENIC POLYPROTEINS OF M. hyopneumoniae, SYNTHETIC GENE ENCODING THE COMPLEX OF IMMUNOGENIC POLYPROTEINS OF M. hyopneumoniae, IMMUNOGENIC COMPOSITION, METHOD FOR PRODUCING A COMPLEX OF IMMUNOGENIC POLYPROTEINS OF M. hyopneumoniae, USE OF A COMPOSITION BASED ON A COMPLEX OF IMMUNOGENIC POLYPROTEINS OF M. hyopneumoniae

(71) Applicant: OURO FINO SAUDE ANIMAL LTDA, Distrito Industrial de Cravinhos, SP (BR)

(72) Inventors: Celso Raul Romero Ramos, Ribeirao Preto (BR); Miryam Marroquin Quelopana, Ribeirao Preto (BR)

(21) Appl. No.: 15/319,652

(22) PCT Filed: May 8, 2015
(86) PCT No.: PCT/BR2015/050056

§ 371 Date: Apr. 17, 2017

(30) Foreign Application Priority Data
Jun. 16, 2014 (BR) BR1020140147276

(51) Int. Cl. A61K 39/02; C07K 14/195; C07K 14/30

(52) U.S. Cl. CPC A61K 39/0241; C07K 14/30; C07K 14/195; A61K 2039/575

(57) ABSTRACT

The present invention relates to a complex of polyproteins containing immunogenic regions derived from the P36, P46, HSP, NrdF, P97, MHP271 e P216 antigens of M. hyopneumoniae, which regions are fused with segments of the Rrp4. Rrp41 and Rrp42 carrier proteins of P. abyssi, forming a single complex. The present invention further provide the following objects: (i) a gene coding the polyprotein complex, which complex is based on the exosome of P. abyssi and carries copies of vaccine antigens of M. hyopneumoniae; (ii) an antigenic composition comprising the polyprotein complex of the invention, optionally one or more auxiliary agents and/or additives and a pharmacologically acceptable excipient; (iii) a method for producing said polyprotein complex; and (iv) uses of said complex and composition in the treatment and prevention of disease caused by M. hyopneumoniae infection.

### TECHNICAL FIELD

This invention refers to a complex of polyproteins with immunogenic activity based on specific polypeptides selected from antigenic proteins of Mycoplasma hyopneumoniae which is the etiological agent of Enzootic Pneumonia of Swine (EPS). More precisely, the present invention relates to a complex of proteins, herein called Exomyc complex, based on exosome proteins of Pyrococcus abyssi carrying vaccine antigens of M. hyopneumoniae.

Other additional objects of this invention provide: (i) an encoding gene of polyproteins comprising a complex based exosome of P. abyssi which carries copies of vaccine antigens of M. hyopneumoniae; (ii) a composition comprising the complex of polyproteins of the invention, optionally one or more adjuvants and/or additives and a pharmaceutically acceptable vehicle; (iii) a process of obtaining said lipoprotein complex, and (iv) uses of said complex of polyproteins and said composition in the treatment and prevention of infections caused by M. hyopneumoniae.

### HISTORY OF THE INVENTION

Mycoplasma hyopneumoniae is the etiological agent of Enzootic Pneumonia of Swine (EPS), a contagious respiratory disease affecting swines worldwide and causing large economic losses.

Commercial vaccines (bacterins—inactivated bacteria) currently used for the control of this disease are not produced in Brazil and have a high cost of production. In addition, immunization with bacterins provides only partial protection and does not prevent the colonization of the epithelium by M. hyopneumoniae (Thacker E L, Thacker B J, Kuhn M, Hawkins P A, Waters W R. (2000) “Evaluation of local and systemic immune responses induced by intramuscular injection of a Mycoplasma hyopneumoniae bacterin to pigs”. Am J Vet Res. 61:1384-1389). The patent document PI0210804, for example, describes a method of treating a disease or disorder caused by M. hyopneumoniae with a vaccine comprising cells of this partially or totally inactivated bacterium or, alternatively polypeptides or immunogenic proteins selected from antigens P46, P65, P97, P102, P70, P50 and P44 of M. hyopneumoniae. In this same line of proposals, the document WO200665841 can be cited.

Vaccine antigens are being tested in different immunization systems using the technology of recombinant DNA. However, such antigens individually provides only partial protection (Simionatto S, Marchioro S B, Maes D, Dellagostin O A. (2013) “Mycoplasma hyopneumoniae: from disease to vaccine development”. Vet Microbiol. 165:234-242). An example of a vaccine based on antigens mixtures is disclosed in EP1245677 reporting the mixture of antigens encoded by the mhp3 gene of M. hyopneumoniae at least one polypeptide selected from P46, P65, P97, P102 of M. hyopneumoniae.

One of the main vaccine antigens tested so far is a protein called P97R1, containing the binding site to cilia of the main adhesion of Mycoplasma hyopneumoniae, the P97 (Wilton J L, Scarman A L, Walker M J, Djordjevic S P. (1998) ”Reiterated repeat region variability in the ciliary adhesin gene of Mycoplasma hyopneumoniae”. Microbiology. 144:1931-1943). Adherence to cilia is an important event for the pathogenicity of M. hyopneumoniae. Bacterial vaccine does not induce antibodies against P97, which would explain the fact that immunization with bacterins does not prevent the initial colonization of the lung tissue (Chen A Y, Fry S R, Daggard G E, Mukkur T K. (2008) “Evaluation of immune response to recombinant potential protective antigens of Mycoplasma hyopneumoniae delivered as cocktail DNA and/or recombinant protein vaccines in mice”. Vaccine. 26:4372-4378). An alternative to improve performance and effectiveness of a vaccine based on antigen P97 is described in the patent document PI0603619 which describes a process of vaccine preparation of recombinant subunit (rLTBFM) against PES, comprising the melting of B subunit of E. coli enterotoxin (LTB, adjuvant) with the repeating region R1 of adhesin P97 of M. hyopneumoniae (R1, immunogen).

The immunization with various antigens prepared separately, either as protein or as DNA vaccine, was able to induce the immune response both humoral and cellular against each of the proteins tested, that is, P42, P97R1, P46 and NrdF (Chen A Y, Fry S R, Daggard G E, Mukkur T K. (2008) “Evaluation of immune response to recombinant potential protective antigens of Mycoplasma hyopneumoniae delivered as cocktail DNA and/or recombinant protein vaccines in mice”. Vaccine. 26:4372-4378); as weel as P37, P42, P46 and P95 (Galli V, Simionatto S, Marchioro S B, Fisch A, Gomes C K, Conceicão F R, Dellagostin O A. (2012) “Immunization of mice with Mycoplasma hyopneumoniae antigens P37, P42, P46 and P95 delivered as recombinant subunit or DNA vaccines”. Vaccine. 31:135-140).

However, the production of separate antigens is still an impediment for industrial production of a vaccine containing all these antigens, because it would be necessary to carry out several individual fermentations for each antigen, combining the antigens obtained in a final pool and only then use this pool for immunization. This process is extremely complex and costly, invalidating large-scale production.

Recently, it was proposed the merging of different antigens of M. hyopneumoniae with protective capacity at adjuvant unit (Marchioro, S. B. (2013) “Evaluation of vaccines against Mycoplasma hyopneumoniae in swines and mice”. Thesis (Doctorate. Post Graduation Program in Biotechnology. Federal University of Pelotas). In said work, an encoding gene of a polyprotein was synthesized, which contains the antigens of M. hyopneumoniae P97R1, P42 and Nrdf attached to the protein rLTB (subunit B of thermolabile enterotoxin of Escherichia coli, used as adjuvant). The immunization with this recombinant protein stimulated the formation of antibodies against each of the three antigens of mycobacteria, showing it is possible to stimulate an immune response against different antigens expressed as a single protein (polyprotein).

Thus, the construction of synthetic genes encoding more than one vaccine antigen of M. hyopneumoniae represents the possibility of obtaining a recombinant vaccine effective against this bacterium, at low cost and with a simple fermentation process.

### SUMMARY OF THE INVENTION

The present invention reveals new polyproteins containing immunogenic regions derived from antigens P36, P46, HSP, NrdF, P97, MHP271 and P216 of M. hyopneumoniae, such regions that are fused to segments of the carrier proteins Rrp4, Rrp41 and Rrp42 of exosome of Pyrococcus abyssi.

According to a first aspect of the present invention, it is provided a complex of three polyproteins characterized by amino acid sequences herein identified as SEQ ID NO:26, SEQ ID NO:27 e SEQ ID NO:28.

According to a second aspect of the present invention, there are provided amino acid sequences having conservative substitutions in the sequences SEQ ID NO:26, SEQ ID NO:27 and SEQ ID NO:28, as well as immunogenic fragments thereof comprising conservative substitutions.

According to a third aspect of the present invention, it is provided a sequence of nucleotides—SEQ ID NO:25—comprising the encoding synthetic gene to the amino acid sequences of three polyproteins comprehended by Complex of Exomyc Proteins of the present invention, nominally, the sequences SEQ ID NO:26, SEQ ID NO:27 e SEQ ID NO:28, or of a sequence of nucleotides having at least 75% of identity related to SEQ ID NO:25.

In a fourth aspect, the present invention relates to na antigenic composition comprising (i) at least two polypeptides of complex of polyproteins, comprising SEQ ID NO:26, SEQ ID NO:27 and SEQ ID NO:28, their analogs, derivative or immunogenic fragments thereof, including conservative substitutions in SEQ ID NO:26, SEQ ID NO:27 and SEQ ID NO:28, (ii) optionally, additives or potentiating substances of immunogenic activity of polyproteins and (iii) a pharmacologically acceptable vehicle.

In a fifth aspect, the present invention refers to a process comprising the steps of: (i) inserting a fusion gene comprising antigenic and stable segments derived from antigens P36, P46, HSP, NrdF, P97, MHP271 and P216 of M. hyopneumoniae with carrier proteins Rrp4, Rrp41 and Rrp42 of P. abyssi in an expression region of a plasmid, preferably the pMRK between sites Ndel and Hind III; (ii) transforming the host cell, preferably E. coli, with said plasmid transformed by insertion of sequences: SEQ ID NO:1 (derived from P36 antigen), SEQ ID NO:2 (derived from P46 antigen), SEQ ID NO:3 (derived from HSP antigen (P42)), SEQ ID NO:4 (derived from NrdF antigen), SEQ ID NO:5 (derived from P97 antigen), SEQ ID NO:6 (derived from MHP271 antigen), SEQ ID NO:7 and SEQ ID NO:8 (derived from P216 antigen), SEQ ID NO:9 (derived from Rrp4 carrier protein), SEQ ID NO:10 (derived from Rrp41 carrier protein) and SEQ ID NO:11 (derived from Rrp42 carrier protein); and (iii) expressing the fused heterologous protein from host cell to obtain polyproteins of sequences SEQ ID NO:26, SEQ ID NO:27 e SEQ ID NO:28, or analogs thereof having conservative substitutions, or antigenic fragments thereof having conservative substitutions.

Also, according to a sixth aspect, the invention refers to the use of (a) complex of polyproteins of SEQ ID NO:26, SEQ ID NO:27 and SEQ ID NO:28, analogs, derivatives or antigenic fragments thereof, including conservative substitutions in SEQ ID NO:26, SEQ ID NO:27 and SEQ ID NO:28, and/or (b) an antigenic composition comprising said polyproteins of SEQ ID NO:26, SEQ ID NO:27 and SEQ ID NO:28, analogs, derivatives or immunogenic fragments thereof, including conservative substitutions in the SEQ ID NO:26, SEQ ID NO:27 and SEQ ID NO:28 in the treatment or prophylaxis of medical and/or veterinary conditions related to infection by M. hyopneumoniae.

### BRIEF DESCRIPTION OF FIGURES

FIG. 1 shows the characterization of P36 antigen of M. hyopneumoniae. FIG. 1A presents the amino acids sequence of this protein; highlighting in gray ( ) the sequence selected to include in the complex of the present invention. FIG. 1B illustrates “ribbons” Diagram of P36 structural model of M. hyopneumoniae.

FIG. 2 shows the characterization of P46 antigen of M. hyopneumoniae. FIG. 2A presents the amino acids sequence of this protein, showing the peptide segment highlighted in gray ( ) which was selected to include in the complex of the present invention. FIG. 2B illustrates the “ribbons” Diagram of P46 structural model of M. hyopneumoniae.

FIG. 3 shows the characterization of P42 antigen (HSP) of M. hyopneumoniae. FIG. 3A presents the amino acids sequence of this protein, showing the peptide segment highlighted in gray ( ) which was selected for inclusion in the complex of the present invention, the sequence used in other works as P42 antigen is underlined ( ). FIG. 3B illustrates the “ribbons” Diagram of structural model of HSP70 protein of M. hyopneumoniae, (HSP70 M. hyopneumoniae), the P42 antigen, derived from HSP70, used in other works (P42 antigen), and, in the lower part of FIG. 3B, the region of HSP70 of M. hyopneumoniae which was selected for inclusion in the complex of the present invention (protein chosen to the complex).

FIG. 4 shows the characterization of NrdF antigen (corresponding to enzyme Ribonucleoside-diphosphate reductase) of M. hyopneumoniae. FIG. 4A presents the amino acids sequence of this protein. The sequence used in other works as being the NrdF antigen is highlighted in grey ( ). For inclusion in the complex of the present invention, it was added to the sequence of NrdF antigen, highlighted in grey ( ), the outlined amino acids (∥ ∥), in order to improve the stability of antigen. FIG. 4B illustrates the “ribbons” Diagram of whole NrdF protein (whole NrdF protein of M. hyopneumoniae), of NrdF antigen used in other works (NrdF antigen) and protein derived from NrdF of M. hyopneumoniae which was selected for inclusion in the complex of the present invention (sequence chosen for complex).

FIG. 5 shows the characterization of antigen P97 de M. hyopneumoniae. FIG. 5A presents the amino acids sequence of this protein, showing the segments highlighted in gray ( ) and marked with underline ( ), corresponding to the sequences R1 and R2, respectively, responsible for the function of adhesion to the epithelial tissue. Segments containing these sequences, and including the outlined segment (∥ ∥), were selected for inclusion in the complex of the present invention. FIG. 5B illustrates the analysis of C-terminal sequence of P97 protein in the PROTEAN program, showing the prediction of secondary alpha-helix structures and antigenicity index. FIG. 5C shows the amino acids sequence containing the regions R1 and R2, which was chosen for inclusion in the complex of invention and the FIG. 5D illustrates the analysis of this sequence, by the PROTEAN program.

FIG. 6 shows the characterization of antigen MHP271 de M. hyopneumoniae. FIG. 6A presents the amino acids sequence of this protein, showing the sequences R1 and R2, responsible for adhesion, which are outlined (∥ ∥) or underlined ( ) respectively. The segments highlighted in grey ( ) were chosen for their inclusion in the protein complex of the present invention. FIG. 6B illustrates the analysis of the sequence chosen for MHP271 protein in the PROTEAN program, showing the prediction of secondary alpha-helix structures and antigenicity index.

FIG. 7 shows the characterization of P216 antigen of M. hyopneumoniae. FIG. 7A shows the sequence of P216 protein; in which the region of adhesion to the platelets is highlighted in grey ( ). Inside this region, two antigen fragments were chosen with alpha-helix structure (predicted by the PROTEAN program) to be used as spacers (“linkers”), such fragments are marked with dashed ( ) or are underlined ( ). FIG. 7B shows the derivatives segments of this antigen which were selected to be included in complex of the present invention as “linkers”.

FIG. 8 presents the organization of genes of P. abyssi exosome.

FIG. 9 shows the complex surface diagram of P. abysssi exosome formed by the Rrp4, Rrp41 and Rrp42 proteins.

FIG. 10 shows the sequence diagram of fusions that comprise the assembly/construction of complex of the invention, as well as polyproteins of Exomyc complex of the invention itself.

FIG. 11 illustrates the cloning strategy of the Exomyc complex from pBluescript KS plasmid (pBSK) containing the synthetic gene in the pMRK expression vector by the sites of Ndel and HindIII.

FIG. 12 illustrates obtaining the antigens of M. hyopneumoniae separately recombinantly. FIG. 12A shows the strategies for performing the antigens subcloning from the initial synthetic gene of the Exomyc complex. Plasmids were constructed for expressing the following recombinant proteins: 6× His-SUMO-P36, 6× His-SUMO-MHP271, 6× His-P46, 6× His-SUMO-P97R1 R2, 6× His-HSP and 6× His-SUMO-NrdF. FIG. 12B shows the purified recombinant antigens: M.—Denotes the molecular mass marker, 1.—Denotes the protein 6× His-SUMO-P36, 2.—Denotes the protein 6× His-SUMO-MHP271, 3.—Denotes the protein 6× His-P46, 4.—Denotes the protein 6× His-SUMO-P97R1 R2, 5.—Denotes the protein 6× His-HSP, 6.—Denotes the protein 6× His-SUMO-NrdF. An asterisk indicates the migration of protein 6× His-SUMO-MHP271.

FIG. 13 illustrates the expression of polyproteins of EXOMYX complex in Escherichia coli strain BL21 (DE3) transformed with the pMRK-EXOMYC plasmid. The bacterial lysate was incubated for 30 minutes at 25 and 100° C., cooled on ice for 5 minutes and subjected to centrifugation for 15 minutes at 12000×g. M.—Denotes the molecular mass marker; NI.—denotes the non-induced material; I.—denotes the culture induced with IPTG (Isopropial thio-galactoside) or Lactose, Solu.—denotes the soluble protein after centrifugation. Pellet.—denotes the sedimented protein during centrifugation.

FIG. 14 illustrates the purification result of EXOMYX complex expressed in Escherichia coli, by tangential filtration. M.—Denotes the molecular mass marker; 1.—denotes the analysis of 2 μL of purified protein; 2.—denotes the analysis of 4 μL of purified protein.

FIG. 15 illustrates the analysis by ELISA of anti-P46 antibodies during the immunization of porcines with vaccines containing the Exomyc complex.

FIG. 16 illustrates the analysis by ELISA of anti-HSP antibodies during the immunization of porcines with vaccines containing the Exomyc complex.

FIG. 17 illustrates the analysis by ELISA of anti-P97R1 R2 antibodies during the immunization of porcines with vaccines containing the Exomyc complex.

FIG. 18 illustrates the analysis by ELISA of anti-NrdF antibodies, C-terminal portion during the immunization of porcines with vaccines containing the Exomyc complex.

FIG. 19 illustrates the analysis by Western Blot of anti-P36 antibodies during the immunization of porcines with vaccines containing the Exomyc complex. Group A.—Animals immunized with 1 ml of formulation (200 μg of Exomyc complex); Group B.—Animals immunized with 2 ml of formulation (400 μg of Exomyc complex).

FIG. 20 shows image examples of lungs of animals sacrificed after 140 days of a single immunization, few (animal A6, of group immunized with 1 ml of the formula) or no injury (animal B7, of group immunized with 2 ml of formula).

### DETAILED DESCRIPTION OF THE INVENTION

As described above, a first aspect of the invention is obtaining the three polyproteins corresponding to the complex based on P. abyssi exosome carrying copies of vaccine antigens of M. hyopneumoniae. To reach this goal, firstly the antigenic proteins of M. hyopneumoniae were selected, candidates to provide protection against infection caused by this bacterium. After the selection of candidate proteins, the great challenge was to choose the sequence fragments of each of these proteins that maintain the immunogenic activity and stability of the final polyprotein and that were able to merge with the P. abyssi exosome to obtain the aimed complex.

The term plasmid pMRK, as used herein, means a vector derived from the pAE vector, in which the following changes were performed: (a) substitution of the antibiotic resistance gene; and (b) introduction of the “parent” partition sequence of plasmid pSC101 (this paragraph was removed from the application of “Vectors” to give descriptive sufficiency to the employment of pMRK vector in obtaining the recombinant protein of the invention (EXOMYC).

The term “polyprotein” is used herein to name the only polypeptide sequence containing sequences of different proteins. The terms used as “complex of proteins” and “Exomyc complex of proteins” are used herein as synonyms with each other and means the complex resulting of binding or coupling of polyproteins identified as SEQ ID NO:26, SEQ ID NO:27 and SEQ ID NO:28, including the fragments and the conservatively modified variants having at least 85% of identity with the SEQ ID NO:26, SEQ ID NO:27 and SEQ ID NO:28. Said synonymous terms also cover the meaning of polyproteins encoded by synthetic gene of the invention identified by SEQ ID NO:25, or by a synthetic gene having at least 75% of identity with respect to SEQ ID NO:25.

Selection of Candidate Antigenic Proteins of M. hyopneumoniae

The present invention is based on the construction of a complex of proteins present in the P. abyssi exosome, said construction being formed from three copies of each specific antigen of M. hyopneumoniae. The study that resulted in the polyprotein construction of the invention was initiated by selection of antigens defined below:
P36 L-lactate dehydrogenase, whole protein.
P46 46kd surface antigen, whole protein.
HSP-2C two domains of C-terminal extreme of HSP70.
NrdF Three alpha-helices of C-terminal extreme of NrdF protein (Ribonucleoside-diphosphate reductase).
P97 R1 and R2 Fragments of P97, responsible for binding to cilia.
MHP271 R1 and R2 Fragments of MHP271, responsible for binding to cilia.
P216 Two segments of alpha-helices of domain of binding to platelets of P216, used as linkers (spacers between the proteins).

This selection was performed based on extensive bibliographic review. The amino acids sequences of antigens were obtained from GenBank, looking to select those belonging to pathogenic strains of M. hyopneumoniae.

P36 (Swiss Prot: POCOJ2.1)

The P36 antigen is a cytoplasmic enzyme, lactate dehydrogenase that is highly immunogenic. Serum from infected animals recognizes with great intensity this antigen, why initially it was used for the diagnostic test, due to its specificity for M. hyopneumoniae (Stipkovits et al., Stipkovits L, Nicolet J, Haldimann A, Frey J. (1991) “Use of antibodies against the P36 protein of Mycoplasma hyopneumoniae for the Identification of M. hyopneumoniae strains”. Mol Celi Probes. 5:451-457). In pigs experimentally infected with M. hyopneumoniae, two peaks were detected in the production of antibodies against P36 (Frey J, Haldimann A, Kobisch M, Nicolet J. (1994) “Immune response against the L-lactate dehydrogenase of Mycoplasma hyopneumoniae in enzootic pneumonia of swine”. Microb Pathog. 17:313-322). The first, a relatively low response was detected in the period between 5 and 10 weeks after infection, time that the clinical signs are apparent, including lung lesions. This response anti-P36 occurred simultaneously with a strong response of antibodies against membrane antigens of M. hyopneumoniae extracted with Tween 20. The second peak response happens from the twelfth week after infection, when the signs of the disease and the infectious agent have disappeared and do not have more antibodies against the proteins of membrane (Frey et al., 1994).

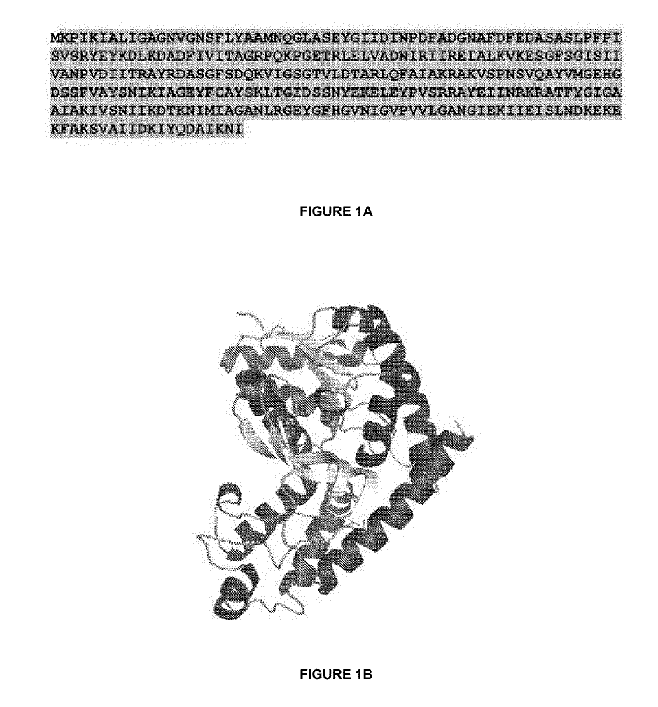

The amino acids sequence do P36 antigen, as filed in the database SwissProt (access P0C0J2.1) (shown in FIG. 1A) It was subjected to modeling the three-dimensional structure in the automatic mode on the server SWISS-MODEL (http://swissmodel.expasy.org). The chosen mold was the PDB 1 EZ4 structure which corresponds to the crystal structure of L-lactate dehydrogenase of Lactobacillus pentoses. The obtained structural model is shown in FIG. 1B.

In functional status, the lactate dehydrogenase forms a tetramer, which can dissociate depending on the pH and the presence of urea (Mayr U, Hensel R, Kandler O. (1980) “Factors affecting the quaternary structure of the allosteric L-lactate dehydrogenase from Lactobacillus casei and Lactobacillus curvatus as investigated by hybridization and ultracentrifugation”. Eur J Biochem. 1 10:527-538). The tetramers are formed in pH 5.0-5.5, whereas the monomeric form prevails to basic pH values. This fact was taken into account to not promote protein aggregates of the assembled structure that forms the complex of the present invention.

As shown in FIG. 1B, this structure has the characteristics of a globular, compact conformation, without domains. In the immunization tests, this protein was used in its complete form, for this reason, the amino acids sequence corresponding to the complete P36 antigen, less the methionine of the beginning of translation, was selected to be included in the complex of the present invention. Thus, this sequence was renamed herein as SEQ ID NO:1 (Table 1).

2. P46 (GenBank: AAV27597.1)

P46 is a surface protein of M. hyopneumoniae, highly antigenic. Similarly to the P36, P46 is used in diagnostic tests to specific identification of this mycoplasma ((Futo S, Seto Y, Okada M, Sato S, Suzuki T, Kawai K, Imada Y, Mori Y. (1995) “Recombinant 46-kilodalton surface antigen (P46) of Mycoplasma hyopneumoniae expressed in Escherichia coli can be used for early specific diagnosis of mycoplasmal pneumonia of swine by enzyme-linked immunosorbent assay”. J Clin Microbiol. 33:680-683; Cheikh Saad Bouh K, Shareck F, Dea S. (2003) “Monoclonal antibodies to Escherichia coli-expressed P46 and P65 membranous proteins for specific immunodetection of Mycoplasma hyopneumoniae in lungs of infected pigs”. Clin Diagn Lab Immunol. 10:459-468; Okada M, Asai T, Futo S, Mori Y, Mukai T, Yazawa S, Uto T, Shibata I, Sato S. (2005) “Serological diagnosis of enzootic pneumonia of swine by a double-sandwich enzyme-linked immunosorbent assay using a monoclonal antibody and recombinant antigen (P46) of Mycoplasma hyopneumoniae”. Vet Microbiol. 105:251-259).

In proteome studies with respect to membrane proteins of M. hyopneumoniae, the protein P46, as well as the ciliary adhesin P97, was identified in different types of extracts, confirming its association with the membrane of mycoplasma (Tavares, CP. (2010) “Análise proteômica de proteins de membrana de Mycoplasma hyopneumoniae e Mycoplasma Synoviae”. Dissertation (Masters in Biochemistry), Post Graduation Course in Biochemistry, Federal University of Santa Catarina, Florianópolis, Brazil).

In the joint immunization with many antigens in the form of DNA vaccines, only P46 could induce humoral response, whereas all other antigens, in this type of immunization, induced cellular response.

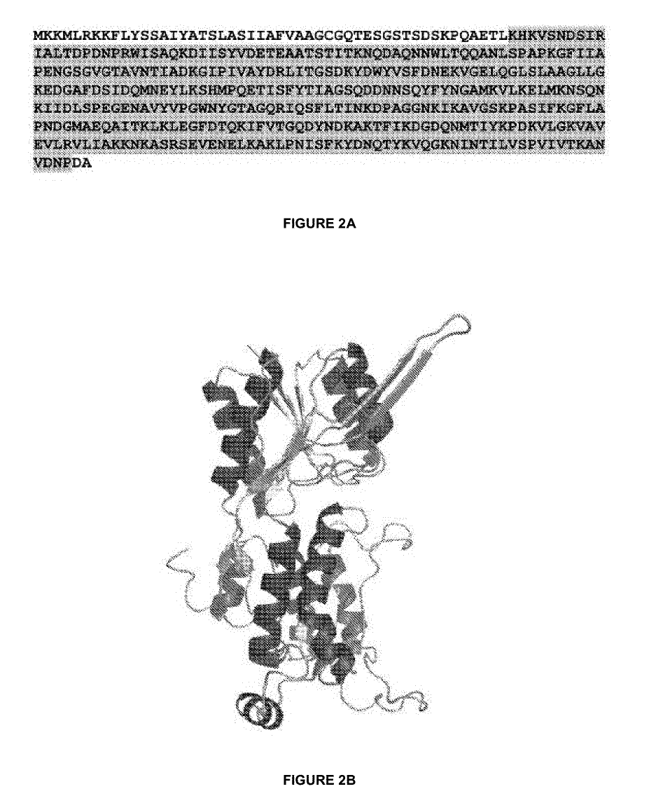

The amino acids sequence of P46 antigen, as filed in GenBank (access AAV27597.1) is shown in FIG. 2A. This amino acids sequence was subjected to modeling the three-dimensional structure, in automatic mode on a server SWISS-MODEL. The chosen mold was the PDB 3MA0 structure, corresponding to the Crystal structure of Xylose Binding Protein of Escherichia coli. In other words, the P46 protein participate in the transport of sugars into the cells of the mycoplasma, which represents an important target for the development of vaccine, to the extent that the antibodies generated by the introduction of the P46 antigen in the host may inhibit the transport of sugars, which can be vital to the M. hyopneumoniae.

The model represented in FIG. 2B presents two globular domains and a length of two beta sheets that leave the main structures. The amino acids sequences interweave between the two domains, which makes it impossible to clone only the domains separately. The N-terminal extreme of the model begins in the amino acid number 55. Probably, the first 54 amino acids correspond to the signal sequence, which determines the output of the bacterial cytoplasm, and to an anchoring region in the plasmatic membrane. For the hydrophobic nature of this kind of segments, in the process of the present invention for construction of complex of polyproteins of the invention, it was considered correct no to include these 54 amino acids in the recombinant protein. The amino acids of P46, highlighted in grey ( ) in the protein sequence of FIG. 2A, correspond to the obtained structural model, and however, were chosen to be included in the construction of complex of the invention, passing this segment to be called herein SEQ ID NO:2 (Table 1).

3. P42 (HSP70) (Swiss Prot: Q49539.1)

P42 is derived from a major protein, P65, which is a cytoplasmic protein of M. hyopneumoniae belonging to the family of HSP70 (Proteina de Choque Térmico, Heat Shock Protein, of 70 kDa) (Chou S Y, Chung T L, Chen R J, Ro L H, Tsui P I, Shiuan D. (1997) “Molecular cloning and analysis of a HSP (heat shock protein)-like 42 kDa antigen gene of Mycoplasma hyopneumoniae ”. Biochem Mol Biol Int. 41:821-831). In experiments of immuno-screening with expression libraries constructed with genomic DNA of M. hyopneumoniae, an antigen of 42 kDa was identified (P42) which is a truncated derivative of the HSP70. This protein, P42, It is highly immunogenic and has also been used in diagnostic tests and as a vaccine antigen (Chou S Y, Chung T L, Chen R J, Ro L H, Tsui P I, Shiuan D. (1997) “Molecular cloning and analysis of a HSP (heat shock protein)-like 42 kDa antigen gene of Mycoplasma hyopneumoniae ”. Biochem Mol Biol Int. 41:821-831).

Vaccination with DNA formulation expressing P42 induces an immune response both humoral and cell. Serum from the immunized animals was able to inhibit the growth of M. hyopneumoniae in vitro (Chen Y L, Wang S N, Yang W J, Chen Y J, Lin H H, Shiuan D. (2003) “Expression and immunogenicity of Mycoplasma hyopneumoniae heat shock protein antigen P42 by DNA vaccination”. Infect Immun. 71:1155-1160).

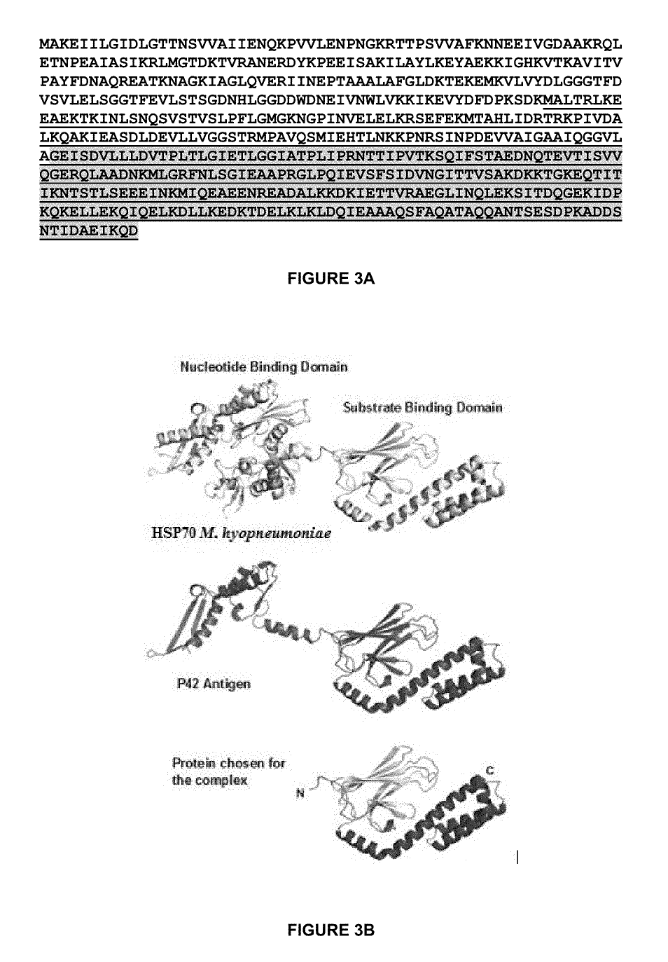

The amino acids sequence do HSP70 antigen of M. hyopneumoniae filed in Swiss Prot (access Q49539.1) is shown in FIG. 3A. This amino acids sequence was submitted to modeling the three-dimensional structure, in automatic mode on a server SWISS-MODEL. The automatically chosen mold was the PDB 1 Q5L structure, the HPS70 of Eschehchia coli, representing the first model in FIG. 3B.

Analyzing amino acids sequence, it was verified that P42 antigen (part of the underlined sequence ( ) in the amino acids sequence of FIG. 3A) contains the truncated NBD domain (Nucleotide Binding Domain), as represented as in the second model in FIG. 3B. The expression, recombinantly, of partial domains can result in proteins with low solubility, which is why it was included in the complex of the present invention, only the SBD domain (Substrate Binding Domain) together with the alpha-helices domain in extreme C-terminal (see model in the lower part of FIG. 3B). These two domains make up a protein 27 kDa. The corresponding amino acids sequence is highlighted in grey ( ) (see FIG. 3A).

The amino acids sequence corresponding to the segment containing said two domains was selected to be included in the complex of the invention, passing this segment to be called herein SEQ ID NO:3 (Table 1).

4. NrdF (Gen Bank AAV27433.1)

During the immuno-screening in an expression library, constructed from the genomic DNA of M. hyopneumoniae using a swine hyperimmune serum, it was identified and cloned a DNA fragment expressing a polypeptide of 11 kDa (Fagan P K, Djordjevic S P, Eamens G J, Chin J, Walker M J. (1996) “Molecular characterization of a ribonucleotide reductase (nrdF) gene fragment of Mycoplasma hyopneumoniae and assessment of the recombinant product as an experimental vaccine for enzootic pneumonia”. Infect Immun. 64:1060-1064). The analysis done by sequencing showed that it is part of a subunit of the Ribonucleotide-diphosphate reductase enzyme of M. hyopneumoniae. Animals immunized by vaccination with this fragment of the protein and challenged with a virulent strain of M. hyopneumoniae had lung disease (measured by the method of mean Goodwin lung score) significantly reduced with respect to control of non-vaccinated pigs (Fagan P K, Djordjevic S P, Eamens G J, Chin J, Walker M J. (1996) “Molecular characterization of a ribonucleotide reductase (nrdF) gene fragment of Mycoplasma hyopneumoniae and assessment of the recombinant product as an experimental vaccine for enzootic pneumonia”. Infect Immun. 64:1060-1064).

Since then, various immunization papers have been published using exactly the same sequence isolated in 1996 (example: Chen A Y, Fry S R, Daggard G E, Mukkur T K. (2008) “Evaluation of immune response to recombinant potential protective antigens of Mycoplasma hyopneumoniae delivered as cocktail DNA and/or recombinant protein vaccines in mice”. Vaccine. 26:4372-4378).

The amino acids sequence of NrdF antigen, as filed in GenBank (access AAV27433.1) is shown in FIG. 4A. This amino acids sequence was subjected to modeling the three-dimensional structure, in automatic mode on the server SWISS-MODEL. The automatically chosen mold was the PDB 4DRO structure, corresponding to the crystal structure of dimanganese (II) NrdF of Bacillus subtilis.

As can be seen, the NrdF of M. hyopneumoniae (FIG. 4B, first model) presents a structure consisting essentially of alpha-helices joined by small “beta-turn” segments.

The sequence isolated in 1996 corresponds to the last two alpha-helices of this structure, wherein, as was selected from a library obtained by random cuts in the genome, the N-terminal extreme of the cloned sequence—FIG. 4B, segment correspondents to the amino acids highlighted in grey ( ) in the sequence of complete NrdF protein shown above—covers only part of one of the alpha-helices.

For the purpose of stabilizing said segment, it was selected the whole alpha-helix. Therefore, the outlined amino acids were added (∥ ∥) in the sequence shown in FIG. 4A, corresponding to the third model of FIG. 4B. The addition of amino acids to complete the alpha helix stabilizes structure to be expressed in the context of complex of the present invention.

Actually, the NrdF antigen corresponds to only a part of a major protein, globular and that does not present domains structure. Nevertheless, analyzing the surface of the protein to be expressed, it appears that this does not display hydrophobic regions which can influence the expression and purification of recombinant protein.

However, the amino acids sequence selected to be included in the complex of the present invention, correspondents to the outlined segment (∥ ∥) and highlighted in grey ( ) in FIG. 4A, is the SEQ ID NO:4 (Table 1).

5. P97 (Gen Bank AAV27763.1)

The p97 adhesin of M. hyopneumoniae, first described in 1995 (Zhang Q, Young T F, Ross R F. (1995) “Identification and characterization of a Mycoplasma hyopneumoniae adhesion”. Infect Immun. 63:1013-1019) (King K W, Faulds D H, Rosey E L, Yancey R J Jr. (1997) “Characterization of the gene encoding Mhp1 from Mycoplasma hyopneumoniae and examination of MhpVs

vaccine potential”. Vaccine. 15:25-35) is considered the main adhesin of mycoplasma, responsible for the adhesion of bacterium to lung cilia.

The specific sequences of P97 protein known as R1 (also called P97R1) and R2 are responsible for this adhesion function (Minion F C, Adams C, Hsu T. (2000) “FM region of P97 mediates adherence of Mycoplasma hyopneumoniae to swine cilia”. Infect Immun. 68:3056-3060) (Jenkins C, Wilton J L, Minion F C, Falconer L, Walker M J, Djordjevic S P. (2006) “Two domains within the Mycoplasma hyopneumoniae cilium adhesin bind heparin”. Infect Immun. 74:481-487).

Immunization experiments with vectored vaccines of whole protein (Shimoji Y, Oishi E, Muneta Y, Nosaka H, Mori Y. (2003) “Vaccine efficacy of the attenuated Erysipelothrix rhusiopathiae YS-19 expressing a recombinant protein of Mycoplasma hyopneumoniae P97 adhesin against mycoplasmal pneumonia of swine”. Vaccine. 21:532-537) or the C-terminal portion, which contains the R1 and R2 sequences (Okamba F R, Arella M, Music N, Jia J J, Gottschalk M, Gagnon C A. (2010) “Potential use of a recombinant replication-defective adenovirus vector carrying the C-terminal portion of the P97 adhesin protein as a vaccine against Mycoplasma hyopneumoniae in swine”. Vaccine. 28:4802-4809) resulted, after the challenge with M. hyopneumoniae, in significant reduction of lung lesions in swine.

Immunogenicity of recombinant vaccines containing the P97R1 sequence and R2 in lab animals is widely characterized in the literature, (Lee S H, Lee S, Chae C, Ryu D Y. (2014) “A recombinant Chimera comprising the R1 and R2 repeat regions of M. hyopneumoniae P97 and the N-terminal region of A. pleuropneumoniae ApxIII elicits immune responses”. BMC Vet Res. 10:43. doi: 10.1 186/1746-6148-10-43; Barate A K, Cho Y, Truong Q L, Hahn T W. (2014) “Immunogenicity of IMS 1 1 13 plus soluble subunit and chimeric proteins containing Mycoplasma hyopneumoniae P97 C-terminal repeat regions”. FEMS Microbiol Lett. doi: 10.1111/1574-6968.12389; Conceicáo F R, Moreira A N, Dellagostin O A. (2006) “A recombinant Chimera composed of R1 repeat region of Mycoplasma hyopneumoniae P97 adhesin with Escherichia coli heat-labile enterotoxin B subunit elicits immune response in mice”. Vaccine. 24:5734-5743; Chen A Y, Fry S R, Forbes-Faulkner J, Daggard G, Mukkur T K. (2006) “Evaluation of the immunogenicity of the P97R1 adhesin of Mycoplasma hyopneumoniae as a mucosal vaccine in mice”. J Med Microbiol. 55:923-929). However, aP97 is an important target for the development of a recombinant vaccine against M. hyopneumoniae.

The immunization with the commercial vaccine (bacterial) does not induce the formation of antibodies against the P97 protein, that would explain the fact that immunization with bacterins does not prevent the initial colonization of lung tissue (Chen A Y, Fry S R, Daggard G E, Mukkur T K. (2008) “Evaluation of immune response to recombinant potential protective antigens of Mycoplasma hyopneumoniae delivered as cocktail DNA and/or recombinant protein vaccines in mice”. Vaccine. 26:4372-4378). Probably, the M. hyopneumoniae in culture does not express the genes which determine the virulence, as P97, whereby no response is generated against this protein, and probably against other important factors in the infection process, in the immunization with bacterin.

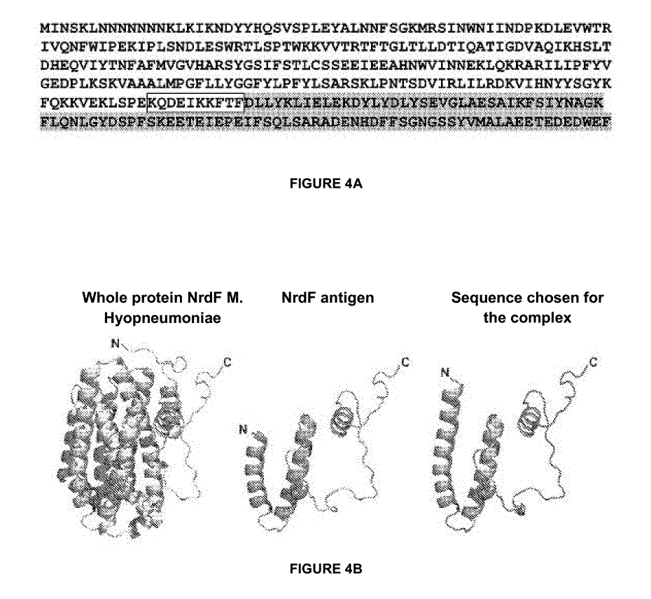

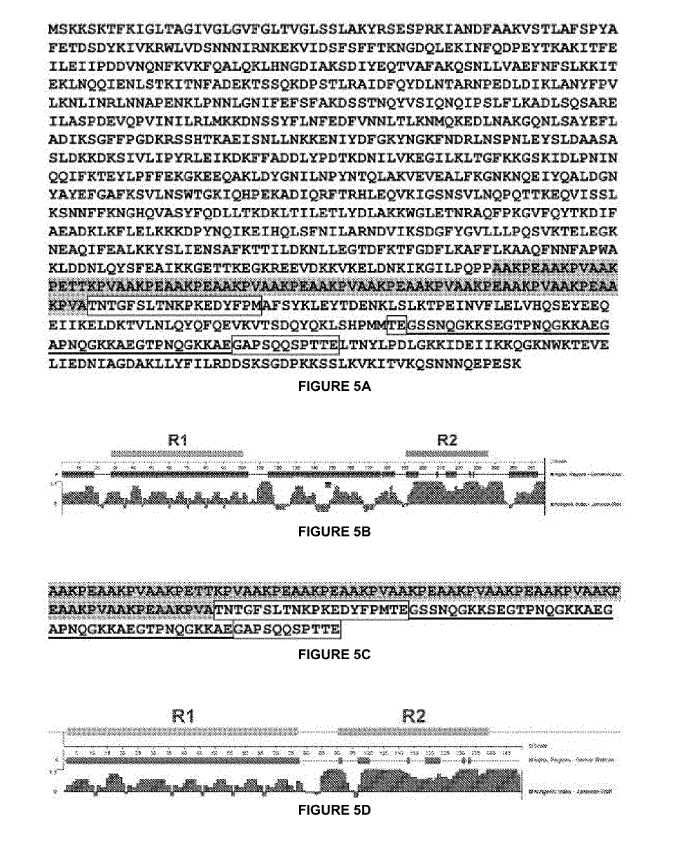

The amino acids sequence of P97 antigen (the P97 gene encodes a protein of 1108 amino acids), as filed in GenBank (access AAV27763.1) is shown in FIG. 5A. The R1 and R2 sequences are highlighted in grey ( ) or marked with underline ( ), respectively. For the construction of complex of the present invention, it was decided to include the R1 and R2 sequences.

As the homologous proteins to P97 with solved structure are not known, it was not possible to model the structure of this protein. For this reason, only the amino acids sequence was analyzed using the PROTEAN program of Lasergene. This program allows to analyze and to visualize once the secondary structure, hydrophobicity, antigenicity, among other characteristics, using different algorithms.

In FIG. 5B, it is evident that the R1 sequence corresponds to an alpha-helix (rectangles) in the P97 protein. R2 already has small segments in alpha-helix and several flexible “beta-turn”. However, the antigenicity calculation (diagram) indicates that R2 is more antigenic than R1.

For the construction of complex of the present invention, it was employed alpha-helix containing the R1 protein, as well as the region of R2 protein, excluding the intermediate sequences which present poor immunogenicity (see FIG. 5C).

As can be seen in FIG. 5D, the sequences with low immunogenicity were removed with success. The analysis of “surface probability” (PROTEAN) indicates that the R2 region is more exposed than the R1, which is probably due to the fact that R1 has only one alpha-helix, which can help in a greater exposure of this region.

Thus, the amino acids sequence selected to be included in the complex of the present invention, corresponding to the P97R1 R2 segment, is SEQ ID NO:5 (Table 1).

6. MHP271 (Gen Bank AAV27492.1)

The MHP271 protein has recently been characterized as a paralogue of P97, which is able to bind to Heparin and to Fibronectin of lung cilia of swines (Deutscher A T, Jenkins C, Minion F C, Seymour L M, Padula M P, Dixon N E, Walker M J, Djordjevic S P. (2010) “Repeat regions R1 and R2 in the P97 paralogue Mhp271 of Mycoplasma hyopneumoniae bind heparin, fibronectin and porcine cilia. Mol Microbiol. 78:444-458).

There is still no immunization data with this protein, however, the study clearly shows that it is associated to the membrane of M. hyopneumoniae. The R1 and R2 sequences, similar to those described for the P97 antigen, were also characterized and it was verified that they are involved in binding to host proteins, which makes MHP271 an excellent vaccine candidate (Deutscher A T, Jenkins C, Minion F C, Seymour L M, Padula M P, Dixon N E, Walker M J, Djordjevic S P. (2010) “Repeat regions R1 and R2 in the P97 paralogue Mhp271 of Mycoplasma hyopneumoniae bind heparin, fibronectin and porcine cilia. Mol Microbiol. 78:444-458).

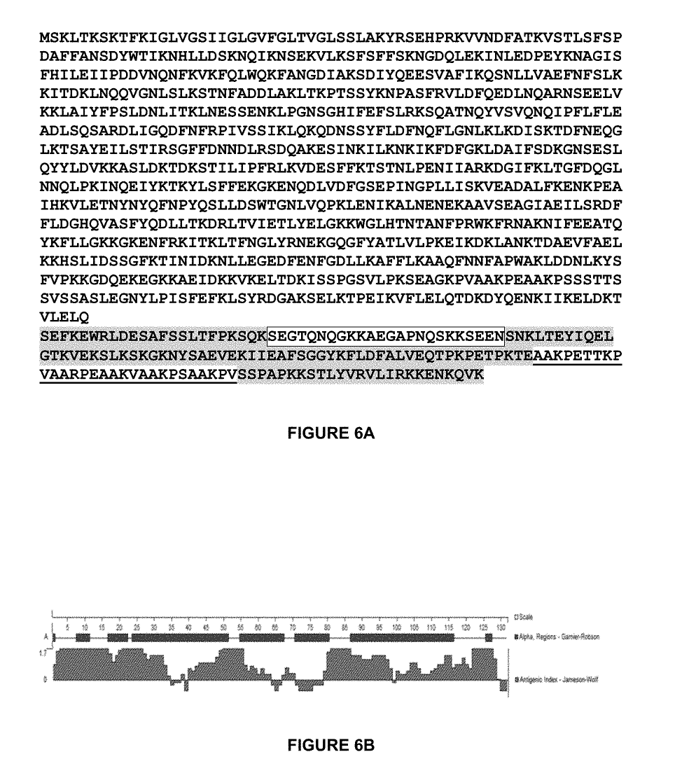

The amino acids sequence of MHP271 antigen (the MHP2717 gene encodes a protein which is also large, 1052 amino acids), as filed in GenBank (AAV27492.1), is the shown in FIG. 6A. In FIG. 6A, the R1 and R2 sequences are those outlined (∥ ∥) or marked with underline ( ), respectively.

The analysis of amino acids sequence using the PROTEAN program of Lasergene, sown in FIG. 6B, indicates that the N-terminal region of this protein, containing R1 and R2 sites, probably corresponds to a domain with secondary structures in alpha-helix. For this reason, all this region was separated with the aim to include the sequences with increased antigenicity, in addition to R1 and R2 proteins.

Thus, a amino acids sequence selected to be included in the complex of the present invention, corresponding to the MHP721-C segment (see FIG. 6C), is SEQ ID NO: 6 (Table 1).

7. P216 (Gen Bank AAZ53862.1)

M. hyopneumoniae has a variety of surface proteins of great molecular mass, with sequences homologous to the P97. However, it is possible to obtain redundant proteins for the function of binding to lung tissue cilia. Even if the P97 is the most abundant, its lock would not prevent colonization by the existence of other adhesins, as MHP271 mentioned above.

P216 is other protein characterized recently. Proteome studies showed that it is associated to the membrane and contributes significantly to the membrane architecture of M. hyopneumoniae, which makes this protein an excellent vaccine candidate (Wilton J, Jenkins C, Cordwell S J, Falconer L, Minion F C, Oneal D C, Djordjevic M A, Connolly A, Barchia I, Walker M J, Djordjevic S P. (2009) “Mhp493 (P216) is a proteolytically processed, cilium and heparin binding protein of Mycoplasma hyopneumoniae ”. Mol Microbiol. 71:566-582). Sequences of R1 type or R2 type were not identified, as occurred in P97 and MHP271 proteins, but it was determined the region of the protein that would be responsible for adhesion (Bogema D R, Deutscher A T, Woolley L K, Seymour L M, Raymond B B, Tacchi J L, Padula M P, Dixon N E, Minion F C, Jenkins C, Walker M J, Djordjevic S P. (2012) “Characterization of cleavage events in the multifunctional cilium adhesin Mhp684 (P146) reveals a mechanism by which Mycoplasma hyopneumoniae regulates surface topography”. MBio. 3(2). pii: e00282-11).

There are no immunization experiments with this protein, However it is known that serum from pigs infected with the P216 of M. hyopneumoniae recognizes this protein (Wilton J, Jenkins C, Cordwell S J, Falconer L, Minion F C, Oneal D C, Djordjevic M A, Connolly A, Barchia I, Walker M J, Djordjevic S P. (2009) “Mhp493 (P216) is a proteolytically processed, cilium and heparin binding protein of Mycoplasma hyopneumoniae ”. Mol Microbiol. 71:566-582).

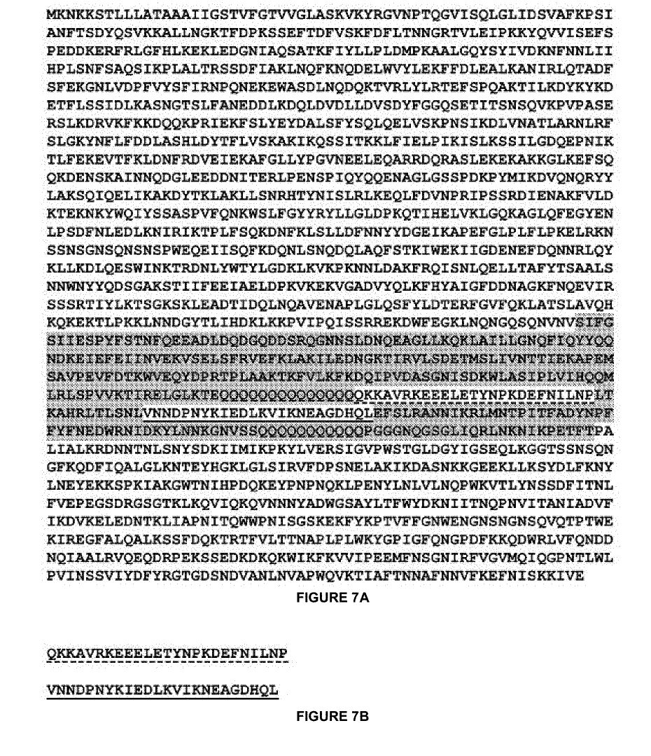

The amino acids sequence do P216 antigen (other protein as large as P97 and MHP271 protein, with 1885 amino acids), as filed in GenBank (access AAZ53862.1), is shown in FIG. 7A).

The sequence highlighted in grey ( ), in FIG. 7A, corresponds to the fragment of P216 responsible for binding to host molecules (Bogema D R, Deutscher A T, Woolley L K, Seymour L M, Raymond B B, Tacchi J L, Padula M P, Dixon N E, Minion F C, Jenkins C, Walker M J, Djordjevic S P. (2012) “Characterization of cleavage events in the multifunctional cilium adhesin Mhp684 (P146) reveals a mechanism by which Mycoplasma hyopneumoniae regulates surface topography”. MBio. 3(2). pii: e00282-11). With the aid of PROTEAN program, in this fragment, two predicted sequences were selected as alpha-helix and with high immunogenicity, marked, respectively, with dash ( ) or with underline ( ) in FIG. 7A and shown separately in FIG. 7B. These sequences were selected to serve as a linker (spacer) among the proteins in the complex of the present invention.

Thus, the amino acids sequences selected to be included in the complex of the present invention, corresponding to the chosen fragments in the sequence of P216 antigen (see FIG. 7B), ate the sequences SEQ ID NO:7 and SEQ ID NO:8 (Table 1).

Table 1 contains the summary of antigenic proteins of M. hyopneumoniae and the amino acids sequences selected from these proteins for inclusion in the Exomyc complex of the present invention, as well as synthetic gene designed to encode said selected amino acids sequences.

TABLE 1
M. hyopneumoniae antigens included in Exomyc complex

| | | | Consideration for | | |
|---|---|---|---|---|---|
| Protein | GenBank | No aa | synthesis | Synthetic Gene | Encoded Protein |
| P36 | SP POCOJ2.1 | 315 | 2-315 | SEQ ID NO: 12 | SEQ ID NO: 1 |
| P46 | BG AAV25597.1 | 419 | 50-417 | SEQ ID NO: 13 | SEQ ID NO: 2 |
| HSP70 | SP Q49539.1 | 600 | 356-600 | SEQ ID NO: 14 | SEQ ID NO: 3 |
| NrdF | GB AAV27433.1 | 353 | 248-353 | SEQ ID NO: 15 | SEQ ID NO: 4 |
| P97 | GB AVV27763.1 | 1108 | 815-907 (R1) | SEQ ID NO: 16 | SEQ ID NO: 5 |
| | | | 979-1031 (R2) | | |
| MHP271 | GB AAV27763.1 | 1052 | 891-1052 | SEQ ID NO: 17 | SEQ ID NO: 6 |
| P216 | GB AVV27492.1 | 1885 | 1272-1296 | SEQ ID NO: 18 | SEQ ID NO: 7 |
| | | | 1309-1332 | SEQ ID NO: 19 | SEQ ID NO: 8 |

Pyrococcus abyssi Exosome

Pyrococcus abyssi is a hyperthermophilic archaebacterium, who lives near hydrothermal vents on the ocean floor. It supports temperatures above 100° C. and pressure up to 200 atmospheres, not representing, however, danger to the health of humans, plants or animals.

To withstand such high temperatures and pressures, the evolution made P. abyssi to have equally stable proteins resistant to denaturation by temperature or pressure. A derivative complex of P. abyssi, called exosome, was characterized extensively (Ramos C R, et al. (2006) The Pyrococcus exosome complex: structural and functional characterization. J Biol Chem. 281 :6751-6759; Navarro M V et al. 2008. Insights into the mechanism of progressive RNA degradation by the archaeal exosome. J Biol Chem. 283:14120-14131).

The complex consists of three copies of three different proteins (9 subunits in total). Its structural characterization allowed the identification of two amino acids in one of the subunits which is the most important for the enzymatic activity of the complex (Navarro M V et al. 2008. Insights into the mechanism of progressive RNA degradation by the archaeal exosome. J Biol Chem. 283:14120-14131). Substitutions of said amino acids by others inhibit the enzymatic activity of this complex, which participates in cell RNA processing and degradation.

The ease of assembly of this complex in the cytoplasm of E. coli, combined with its high stability and solubility, suggests that exosome can be used as peptides and proteins carrier, because the three-dimensional structure shows that the amino and carboxyl extremes of the subunits protrude out of the complex.

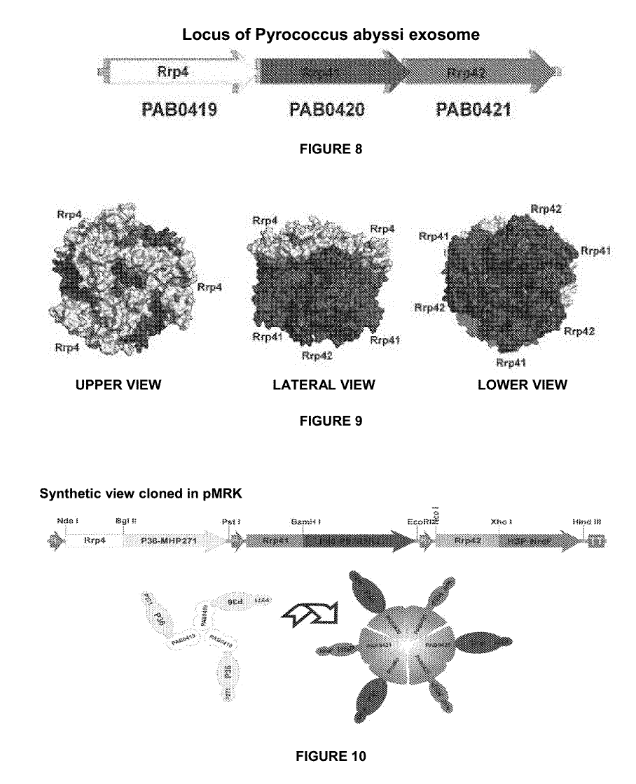

Genes encoding proteins that comprise the complex of Pyrococcus abyssi GE5 exosome, are located at a single locus in the genome of this archaebacterium (Koonin E V et al. (2001) “Prediction of the archaeal exosome and its connections with the proteasome and the translation and transcription machineries by a comparative-genomic approach”. Genome Res. 11:240-252) (see FIG. 8). This locus contains three genes, PAB0419, PAB0420 and PAB0421 encoding the Rrp4, Rrp41 and Rrp42 proteins, respectively.

The complex of proteins comprising the P. abyssi exosome is illustrated in FIG. 9. The Rrp41 and Rrp42 proteins form a hexameric disc (ring) which contains three copies of each protein. Additionally, three copies of the Rrp4 protein bind to one of the disc surfaces, to form a complete exosomic complex (Ramos C R, et al. (2006) “The Pyrococcus exosome complex: structural and functional characterization”. J Biol Chem. 281 :6751-6759).

The complex of archaea has unique characteristics that can be exploited in the development of new generation of vaccines so-called “particles similar to pathogens”, using biomimetics to improve the immune response. These characteristics are:
capacity for specific self-association when expressed in Escherichia coli cells,
high expression, solubility and thermostability of the complex, which facilitates purification and formation of recombinant proteins.
thermostability also confers greater stability of the protein during the formulation and storage of finished products, important aspect in the development of biological products.
ability to carry more than one antigen and immunomodulatory protein, because the amino and carboxyl terminal extremes of the three proteins are exposed to the solvent, as determined structure by crystallographic.

This new methodology represents a major advance regarding the use of unique recombinant antigens in vaccines formulation that can not afford the degree of protection that is achieved with whole organisms such as bacteria and viruses inactivated (Rosenthal J. A. et al. (2014) Pathogen-like particles: biomimetic vaccine carriers engineered at the nanoscale. Current Opinion in Biotechnology (Nanobiotechnology•Systems biology) 28: 51-58).

The expression of proteins in Escherichia coli facilitates the production, due to the complex being resistant to high temperatures, in which proteins from E. coli which is a mesophilic bacteria, completely denature. For example, the treatment at 80° C. for 30 minutes, followed by centrifugation at 5000 g, precipitates almost all the E. coli proteins, allowing to obtain the complex almost pure. The complex is resistant not only at high temperatures, but also to treatments with 8 M urea and the high pressures, conditions in which E. coli protein (or of any mesophylous) denaturates. This quality, plus the fact that the complex has the capacity to carry other proteins and peptides, make the exosome complex is a good candidate to charger of multiple antigens simultaneously. The present inventors are unaware of the use of similar structures for this purpose.

The amino acids sequences of P. abyssi exosome were obtained from GenBank. The amino acids sequences used for the design of synthetic genes correspond to those reported in GenBank to Pyrococcus abyssi strain GE5:

TABLE 2
Proteins of P. abyssi exosome included in Exomyc complex.

| | | No | Considered | Synthetic | Encoded |
|---|---|---|---|---|---|
| Protein | GenBank | aa | for synthesis | Gene | protein |
| Rrp4 | NP_126300.1 | 265 | 2-262 | SEQ ID | SEQ ID |
| | | | | NO: 20 | NO: 9 |
| Rrp41 | NP_126301.1 | 249 | 3-246 | SEQ ID | SEQ ID |
| | | | | NO: 21 | NO: 10 |
| Rrp42 | NP_126302.1 | 274 | 2-274 | SEQ ID | SEQ ID |
| | | | | NO: 22 | NO: 11 |

The Rrp41 protein contains the catalytical site responsible for the RNase activity of the exosome. Since this activity is not desired for the development of vaccines, the amino acids present in the active site were changed, based in published results (Navarro M V et al. 2008. Insights into the mechanism of progressive RNA degradation by the archaeal exosome. J Biol Chem. 283:14120-14131). In this work it is shown that the double mutation R89E and K91 E in Rrp41 abolished the RNase activity of the exosome. In the synthetic gene, R89T and K91 S exchanges were performed, resulting in a smaller exchange in charge of the molecule than that which occurs in the mutations reported in the literature.

Thus, the ssRNA-binding activity of protein Rrp4 (containing the binding domains to RNA S1 and KH) was maintained, as well as the ring structure was maintained (RING) formed by Rrp41-Rrp42 proteins. It is believed that E. coli ssRNA which is co-purified along with the exosome can activate the TLR7 receptor, and thus act as an adjuvant in vaccines based on exosome.

Design of Synthetic Genes for Proteins of M. hyopneumoniae P36, P46, HSP, P97R1 R2, MHP271, segments of P216 protein; and the P. abvssi: Rrp4, Rrp41 and Rrp42

The amino acids sequences used for the design of synthetic genes corresponding to those reported in GenBank as indicated in Tables 1 and 2.

For the design of synthetic genes it took into account the theory known as “Codon Harmonization” (Angov E, et al. (2011) Adjustment of codon usage frequencies by codon harmonization improves protein expression and folding. Methods Mol Biol. 705:1-13). By matching theory codons, codons called rare (which are less commonly used than other codons in a given organism) have a lower amount of tRNAs and play an important role in the folding of proteins. They are located in the “loops” of proteins, among the segments of defined secondary structure. These codons may decrease the rate of protein synthesis at the ribosome, allowing the synthesized sequence to fold before the beginning of synthesis of another protein fragment.

Rare codons in genes encoding the protein shown in Tables 1 and 2, were identified using the ANACONDA program (http://bioinformatics.ua.pt/software/anaconda) fed according to the codon usage table M. hyopneumoniase and P. abyssi, obtained in “Codon Usage Database” (http://www.kazusa.or.jp/codon/).

The location of the amino acids encoded by “rare codons” in the three-dimensional structures obtained for each of the proteins was performed using the Swiss-Pdb-Viewe 4.0.1 program (http://spdbv.vital-it.ch/), or in the predictions of secondary structure by the PROTEAN program, when there is no structural model. In the “harmonization” of synthetic gene, the rare codons were considered encoding amino acids located in the “loops” regions between the secondary structure, alpha-helix or beta-sheet.

For the design of the synthetic gene, it was used the Gene Designer 2.0 program (DNA2.0), manually selecting “rare” codons of E. coli for each of the identified amino acids with ANACONDA programs and located in the structure with Swiss-Pdb-Viewe 4.0.1, in order to improve the folding of proteins. The Gene Designer 2.0 program was fed with the table of codons use for highly expressed proteins in E. coli (Villalobos et al. (2006) Gene Designer: a synthetic biology tool for constructing artificial DNA segments. BMC Bioinformatics. 7:285). In order to improve stability of the gene to be synthesized in “Gene Design”, sequences were avoided which promote recombination, such as “Chi” site, Cre-recombinase, bacterial invertase, Dam metilase, Tn3 Resolvase. In addition to these sequences to be avoided, the sequences of start of restriction enzyme transcription and translation are also avoided, which are used in clonings and are added in the list of sequences that the program avoids in the synthesis of gene (see Table 3).

TABLE 3
List of sequences to be avoided in the design
of synthetic genes of the invention

| Name | Sequence |
|---|---|
| Bacterial Invertase | AAACCAATTTGG |
| Chi Site | GCTGGTGG |
| Cre Recombinase | TACATAC |
| Cryptic Splice Acceptor | YYYYNTAGG |
| Cryptic Splice Donor | MAGGTRAGT |
| Dam Metilase | GATC |
| Lac Repressor Site | GTGGAATTGTGAGCGATA |
| | ACAAT |
| Prokaryotic Ribosome Binding | AGGAGGTNNNNNDTG |
| Site | |
| Resolvase Tn3 | TGTGYNNTA |
| RNA Destabilizing Sequence | ATTTA |
| Shine-Dalgarno | AGGAGGTN(5, 10)DTG |
| Sigma70 Promoter | TTGAC (N)18 TATAAT |

After the generation of sequences, they were subjected to analysis of the positions of rare codons in selected sites, as the CAI (Codon Adaptation Index) using again the ANACONDA program, this time with the codons table of E. coli.

The results were satisfactory with respect to the prediction of expression in E. coli (CAI) and the presence of rare codons, in the chosen sites, trying to simulate the original genes in their respective source organisms (M. hyopneumoniae e P. Abyssi).

The identification of the DNA sequences designed for each protein is shown in Tables 1 and 2.

Planning of Fusions to be Made in Exosomo

In the present invention, proteins of high molecular mass are fused to the exosome. However, it was found to be preferable to fuse at the C-terminal extreme, so as to allow the exosome proteins primarily to fold, by the structure of the exosome, both in the N-terminal and in the C-terminal extreme, which are projected out of the complex of the present invention.

In the planning of fusions to modeling the complex of proteins of the present invention, all chosen amino acids sequences were analyzed in the Prot Param program (http://web.expasy.org/protparam). In Table 4, the results of this analysis are showed.

The chosen amino acids sequences for the M. hyopneumoniae antigens show a low instability index (II), however, they are stable proteins, except the NrdF protein.

TABLE 4
Analysis of physicochemical properties of proteins
selected from the amino acids sequence

| No (*) | Protein | MW (kDa) | pI | I.I (**) | Stability |
|---|---|---|---|---|---|
| 1 | NrdF | 12.3 | 4.14 | 62.12 | Not stable |
| 2 | HSP | 26.8 | 4.53 | 35.98 | Stable |
| 3 | Rrp42 | 30.0 | 4.72 | 37.33 | Stable |
| 4 | P46 | 40.5 | 5.91 | 29.33 | Stable |
| 5 | Rrp41 | 27.6 | 6.26 | 38.47 | Stable |
| 6 | P36 | 34.2 | 7.91 | 25.75 | Stable |
| 7 | Rrp4 | 29.3 | 9.08 | 23.24 | Stable |
| 8 | MHP271 | 14.1 | 9.55 | 38.3 | Stable |
| 9 | P97R1-R2 | 15.0 | 9.65 | 34.07 | Stable |

(*) Proteins arranged according to its Ip (isoelectric point), in increasing order
(**) Instability index

Proteins included in the same fusion were mainly chosen for their similarity in Isoelectric Point. Then, the most important antigens were associated with Rrp41 and Rrp42 proteins that make up the hexameric ring of exosome. It was determined that the largest protein (with improved folding and stability) of M. hyopneumoniae stay directly connected to the exosome proteins, while smaller proteins stay in the C-terminal extreme of the polyproteic fusions:
1. Rrp4-P36-MHP271
2. Rrp41-P46-P97R1 R2
3. Rrp42-HSP-NrdF

Panning of Synthetic gene to express the “Exomyc” Complex of the Invention

After establishing the fusions, it was performed planning to group synthetic genes appropriate to encode the segments/fragments included in the Exomyc complex of the invention. For this planning the following criteria were considered:
1. a single sequence to express three fusions (polyproteins) in the same vector.
2. succession of the fusions identical to the sequence of the gene locus of exosome (FIG. 8).
3. inserting restriction sites that enables expression of the antigens jointly and separately (for the ELISA tests).
4. inserting restriction sites that enables the fusion of other protein or peptide sequences, to the complex of exosome, in such a way that the Exomyc complex of the invention can be used in the formation of similar complexes, including those in which there are constitutive substitutions or that the complexes are obtained in the synthetic genes that, due to “degeneration” of genetic code, encode complexes like Exomyc, including those with constitutive substitutions.

FIG. 10 shows the planning result of the synthetic gene of “EXOMYC” complex of the invention, and a representation of encoded polyprotein forming the complex.

The construction shown in FIG. 10 is designed for the cloning in the pMRKA vector by the Ndel and HindIII sites, so that only the first gene (Rrp4) is under promoter control 17 of vector. For genes Rrp41 and Rrp42, sequences were designed containing the promoter 17, similarly to the co-expression vector pETDuet-1 and pACYCDuet-1 (EMD Millipore), but without the operator sequence of operon lac, present in these commercial vectors: MHP271 stop, Rrp41 Promoter and start (SEQ ID NO:23)

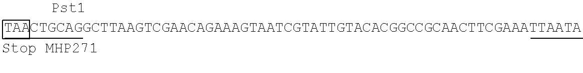

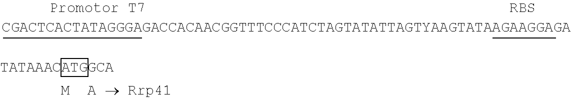

P7R1R2 Stop, Rrp42 promoter and start (SEQ ID NO:24)

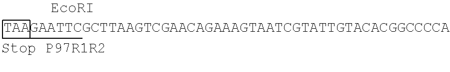

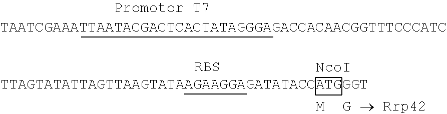

The genes designed for each proteins of Exomyc complex do not contain sequences of translation start or ending, or restriction sites. Similarly to the promoter sequences, small sequences were designed separately to be introduced into the gene of Exomyc complex (SEQ ID NO:25):

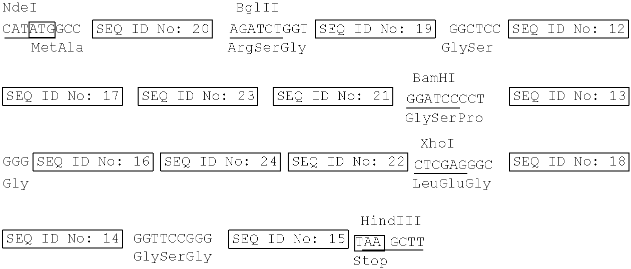

The first inserted fragment contains the cleavage site for the Ndel enzyme, the translation initiation codon (highlighted in the square) for the Rrp41 protein (SEQ ID NO:20) and a codon to the Alanine amino acid.

The second fragment contains the cleavage site for the BgIII enzyme to the cloning in C-terminal fusion of Rrp4 protein, and a codon to the amino acid Glycine, before “Hnker P216-B (SEQ ID NO:19). Between this sequence and the P36 protein (SEQ ID NO:12), it was introduced the dipeptide Glycine-Serine, to give greater flexibility to the protein.

After the sequence encoding the Rrp41 protein (SEQ ID NO:21), has been introduced the cleavage site for the BamHI enzyme for the cloning in the C-terminal fusion of Rrp41 protein, and the codon for the amino acid Proline, before the sequence of P46 protein (SEQ ID NO:13). Between this sequence and the P97R1 R2 protein (SEQ ID NO:16), it was introduced the Glycine codon, to give greater flexibility to the protein.

After the sequence of Rrp42 protein (SEQ ID NO:22), it was introduced the cleavage site to the Xhol enzyme to cloning in C-terminal fusion of Rrp42 protein, and a codon amino acid Glycine, before the sequence of “Hnkef P216-A (SEQ ID NO:18). Between the HSP protein (SEQ ID NO:14) and the NrdF (SEQ ID NO:15), It was inserted tripeptide (Glycine-Serine-Glycine), to increase the flexibility of the molecule. And, finally, it was placed a translation termination site and the cleavage site for HindIII.

The complex of Exomyc proteins of the present invention, encoded by synthetic gene of the invention, characterized by the sequence of nucleotides of SEQ ID NO:25, encodes the polyproteins listed in Table 5.

TABLE 5
Analysis of physicochemical of encoded
polyproteins by gene of Exomyc complex

| Poly- | | | MW | | | | SEQ ID |
|---|---|---|---|---|---|---|---|
| protein | Composition | aa | (kDa) | pL | I.I(*) | Stability | NO: |
| 1 | Rrp4-P216B- | 737 | 80.5 | 9.05 | 27.88 | Stable | SEQ ID |
| | P36-MHP271 | | | | | | NO: 26 |
| 2 | Rrp41-P46- | 765 | 82.7 | 8.81 | 33.31 | Stable | SEQ ID |
| | 97R1R2 | | | | | | NO: 27 |
| 3 | Rrp42-P216A- | 657 | 72.6 | 4.53 | 40.59 | Not | SEQ ID |
| | HSP-NrdF | | | | | stable | NO: 28 |

(*)Instability index

To evaluate the immune response stimulated by Exomic complex for individual antigens, it is necessary to separate cloning thereof in fusion with peptides or proteins different from proteins of the P. abyssi exosome. For this task, specific primers can be used for insertion of restriction sites by PCR amplification, and taking advantage of the existing sites in the synthetic DNA, as illustrated in FIG. 12.

Then, the drawn sequences are provided to this end:

```
T7Rev
SEQ ID NO: 29
5′-TGTGGTCTCCCTATAGTGAGTCG 3′
Gen1BRev (P36 reverse)
SEQ ID NO: 30
          PstI
5′ GGCTGCAGTTAAATGTTTTTAATCGCATCCTG 3′
Gen1CFor (MHP2171 Forward)
SEQ ID NO: 31
            BglII
5′ CCAGATCTCCGAAATCCCAAAAATCTG 3′
Gen2AFor (Rrp41 forward)
SEQ ID NO: 32
          NdeI
5′ AGCATATGGCAGAGAAACCGGAAGG 3′
Gen2BRev (P46 reverse)
SEQ ID NO: 33
            EcoRI
5′ CCGAATTCTTAAGGGTTGTCCACATTCGCTTTGG 3′
Gen2CFor (P97R1 R2 forward)
SEQ ID NO: 34
          BamHI
5′ AAGGATCCGCGAAACCGGAGGCAGCGAAACC 3′
Gen3BRev (HSP reverse)
SEQ ID NO: 35
            HindIII
5′ CCAAGCTTAATCTTGTTTAATCTCCGCGTCG 3′
Gen3CNFor (NrdF Forward)
SEQ ID NO: 36
            XhoI
5′ GGCTCGAGGGGAAACAGGATGAAATCAAAAAATTCAC 3′
```

The synthetic gene of the present invention, comprising the sequence of nucleotides SEQ ID NO:25, encoding of the complex of Exomyc proteins of the invention has the sequence of nucleotides shown in the attached Sequence Listing and integrating the present description. the coding regions of the segments based on the corresponding antigens of M. hyopneumoniae and carrier proteins are listed in Tables 1, 2 and 5.

Considering that it is able to put together the complex shown in FIG. 10, we will have three copies of each polyprotein in complex, with a molecular mass of approximately 700 kDa. It is expected that by the size, the Exomyc complex can act as “particle like pathogen” (Rosenthal J. A. et al. (2014) Pathogen-like particles: biomimetic vaccine carriers engineered at the nanoscale. Current Opinion in Biotechnology (Nanobiotechnology•Systems biology) 28:51-58).

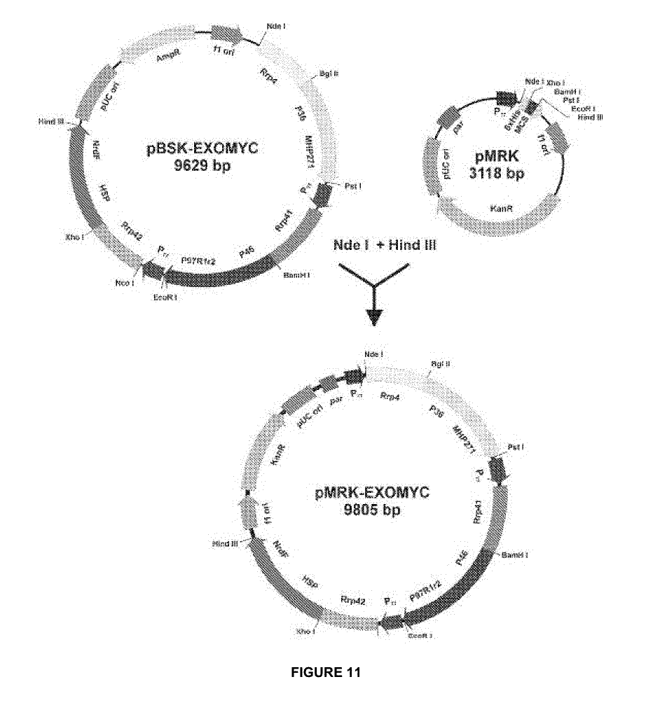

After synthesis of the designed gene, it was cloned into an appropriate vector which contemplates the insertion of a relatively large gene. Numerous expression vectors are commercially available for the construction of encoding synthetic gene of the Exomyc complex of proteins of the invention, such as the vectors of pET series (Novagen) and pRSET (Life Technologies). The person skilled in the art will be able to, from the teachings of the present Invention and from commercially available expression systems, select the appropriate vector for use in the production of the Exomyc complex de proteins of the present invention. More preferably, the synthetic gene of the invention was cloned in the pMRK vector between the Ndel and Hindi 11 sites (FIG. 11).

The expression system for industrial production of Exomyc complex de proteins of the invention can be any commercially available system. An example is the Escherichia coli expression. system, strains BL21 (DE3) and Rosetta (DE3), available from the company Novagen (see Novagen Catalog 2009/2010, pages 121 e 123, available on the Web in www.merckmillichina.com/promart/library/Novagen-pET-E-coli-expression-system.pdf). With the use of this expression system and an expression vector containing the T7 phage promoter, such vector that carries the synthetic gene of the invention, it is possible to obtain the Exomyc complex of proteins of the invention by fermentation as extensive knowledge of the skilled in the art, why there is no need to provide further details of the conditions and steps employed, which may be determined without the need of many experiments. Examples of plasmids for expression of the recombinant protein of the Invention are those of the pET series, such as pRSET, being employed more preferably the PMK vector as defined herein.

It is widely known in the art of chemistry that certain amino acids substitutions can be made in sequences of proteins without affecting the function or activity thereof. Generally, conservative substitutions of amino acids or similar amino acids substitutions can occur naturally or intentionally without modification of the biological activity, for example, immunogenicity of the protein or fused protein. The similar amino acids can be those with size and/or similar charging properties. For example, aspartate and glutamate and isoleucine and valine are pairs of similar amino acids. The similarity between pairs of amino acids can be evaluated by several ways known by the skilled in the art. For example, Dayhoff et al. (1978) in Atlas of Protein Sequence and Structure, Volume 5, Supplement 3, Chapter 22, pp. 345-352, which is incorporated herein by reference, provides frequency tables for amino acids substitutions that can be used as a measure of similarity. The tables of frequency of Dayhoff et al. are based in comparisons of amino acids sequences to proteins having the same function, from a variety of different evolution sources.

Also known is the fact that the “percentage of sequence identity” for polynucleotides and polypeptides is determined by comparing two sequences aligned for optimal manner over a comparison window, wherein the portion of the sequence of polynucleotides or polypeptides, in the comparison window may comprise additions or deletions when compared to a reference sequence (that does not comprise deletions or additions) for optimal alignment of the two sequences. The percentage is calculated by determining the number of nucleic acid base positions or identical amino acid residue which occurs in both sequences to yield the number of matched positions/coincidents by the total number of positions in the comparison window and multiplying the result by 100 to yield the percentage. Optimal alignment of sequences for comparison may be conducted by computerized implementation of known algorithms (for example, GAP, BESTFIT, FASTA and TFASTA presents in Wisconsin Genetics Software Package, Genetics Computer Group (GCG), 575 Science Dr., Madison, Wis., or BlastN and BlastX available from National Center for Biotechnology Information), or by inspection. Sequences are typically compared using BlastN or BlastX with default parameters.

Substantial identity of polynucleotide sequences means that a polynucleotide or gene comprises a sequence having at least 75% of sequence identity, preferably at least 80% and more preferably at least 90% of sequence identity when compared to a reference sequence. Typically, two polypeptides or proteins are considered substantially identical if at least 40%, preferably at least 60%, more preferably 90% and more preferably 95% are identical sequences or have conservative substitutions. The sequences are preferably compared using GAP with default parameters.

It is important to note that the Exomyc complex of proteins of the present invention may contain conservative substitutions, which allow obtaining analogues of said complex, whose analogues preserving the antigenicity of antigen of M. hyopenumoniae which were the basis for the assembly of the complex and, however, such analogues are appropriate to the same uses, should therefore be considered as included within the scope of the present invention. Table 6 shows the examples of said substitutions:

TABLE 6
Examples of conservative substitutions

| Residue | Conservative Substitution |
|---|---|
| Ala | Ser |
| Arg | Lys |
| Asn | Gln, His |
| Asp | Glu |
| Gln | Asn |
| Gly | Pro |
| His | Asn, Gln |
| Leu | Ile, Val |
| Met | Leu, Ile |
| Ser | Thr, Gly |
| Thr | Ser, Val |
| Val | Ile, Leu |

Also included in the present Invention are Exomyc complex proteins fragments of the present Invention that maintain the antigenicity of the full complex as represented by amino acids sequences SEQ ID NO:26, SEQ ID NO:27 and SEQ ID NO:28.

Briefly, the method for producing a polyprotein which corresponds to the fusion and antigenic and stable segments of antigens P36, P46, HSP, NrdF, P97, MHP271 and P216 of M. hyopneumoniae with segments of carrier proteins Rrp4, Rrp41 and Rrp42 of P. abyssi, herein called Exomyc Complex de proteins of the present invention comprises the steps of: (i) inserting a gene fusion in a coding region of a plasmid, preferably the pMRK among the Ndel and Hind III sites; (ii) transforming the host cell, preferably E. coli, with the plasmid; and (iii) expression of the fused heterologous protein from the host cell by methods well known in the art. more particularly, the production method of fused heterologous protein of the present Invention comprises inserting cDNA which is encoded for expression of Exomyc Complex de proteins of the invention, or a fragment thereof, or a conservatively modified variant thereof, in a vector containing the coding information for the production of a carrier protein, preferably the proteins Rrp4, Rrp41 and Rrp42 of the Pyrococcus abyssi archaebacterium. After inserting the vector into the expression system, the fused heterologous protein is expressed by the system.

The present invention also refers to an antigenic composition comprising a polyprotein, corresponding to an immunogenic complex of the invention, or a analogue thereof, as active ingredient and pharmaceutically acceptable non-active ingredients.

The composition of the invention can be in the form of a solid, semi-solid or liquid preparation. Preferably, the preparation is in the liquid form for oral administration, nasal spray or parenteral solution/suspension. The immunogenic polypeptides/polyproteins forming the Exomyc complex of the invention, active ingredient of the composition of the invention, can be in the neutral form or salts, the latter may be addition salts or salts formed with free carboxyl groups, the important being the fact that the said salts are pharmaceutically acceptable. The skilled in the art are able to, from the Invention disclosed herein and the common knowledge of polypeptides/polyproteins salts, those suitable for being included in the composition of the Invention.

According to the invention, the term “pharmaceutically acceptable vehicle” means one or more non-active ingredients which have the function, in the composition, of carrying the active ingredient until the local where its pharmacological activity is required and should include substances suitable for use in humans and animals and be compatible with the active ingredient of the invention, that is, with the complex of immunogenic polyproteins/polypeptides of the invention. Examples of pharmaceutically acceptable vehicles include saline, buffered saline, dextrose, water, glycerol, sterile isotonic solution, and combinations thereof.

The composition of the present invention can comprise, in addition to Exomyc Complex of polyproteins of the invention and pharmaceutically acceptable vehicle, other non-active ingredients, such as, adjuvants, preservatives, diluents, emulsifiers, stabilizers and other pharmaceutically acceptable ingredients which are employed in immunogenic preparations for human and animal use. Non-limiting examples of adjuvants include incomplete Freund's adjuvant, complete Freund's adjuvant, polydispersed acetylated mannan β(1,4) linked, adjuvants of polyoxyethylene-polyoxypropylene copolymer, modified lipid adjuvants, adjuvants derived from saponine, dextran sulfate, aluminum hydroxide and aluminum phosphate.

The composition of present invention can be administered to an animal, including man, by means known in the art. For example, the composition may be administered parenterally, for example, subcutaneous, intraperitoneal, or intramuscular, or by oral route or nasal spray and may be administered in one or more daily doses.

The amount of the composition of the invention administered to an animal varies according to the species of animal. For cattle, the amount of Exomyc complex of protein per dose may vary from 50 to 500 micrograms. The dose of the composition of the Invention to be administered to the subject contains the Exomyc Complex of proteins of the invention, or conservatively modified variant or immunogenic fragments thereof, in a amount smaller than 1.0 mg, preferably at most 0.5 mg, more preferably still in the range of 0.2 until 0.4 mg. The skilled in the art, based on the teachings of the present invention may be able to determine by routine testing the amount of the Exomyc Complex of proteins of the invention, or a conservatively modified variant or immunogenic fragments thereof which will be necessary to develop an adequate immune response to said protein by the animal.

The following examples are provided which illustrate and represent specific embodiments of the invention. The following examples should not be construed as limiting the present Invention.

### EXAMPLES

### Example 1

### Cloning of Synthetic Gene of the Complex Exomyc in the pMRK Expression Vector

The sequence of the designed gene, corresponding to SEQ ID NO:25, encodes sequences SEQ ID NO: 26, SEQ ID NO: 27 and SEQ ID NO 28 which comprise the EXOMYC complex of the present invention.

The synthetic gene was cloned into a vector without the multiple cloning site derived from pBlueScript II SK cut with Sma called pBSK-EXOMYC. The sequence of synthetic gene (SEQ ID NO:25) was confirmed by sequencing by technique known by the skilled in the art.

In one of the embodiments of the present invention, the expression of recombinant proteins was performed in the pMRK expression vector of E. coli. FIG. 11 shows the cloning strategy. The synthetic gene was cut from plasmid pBSK-IniOF using the Ndel and Hind III enzymes and cloned in the corresponding sites of pMRK vector. The resulting plasmid was named pMRK-EXOMYC (FIG. 11). The successful cloning was verified by restriction analysis, as widely known in the art.

### Example 2

### Cloning and Expression of M. hyopneumoniae Antigens Included in the EXOMYC Complex

For immunological characterization of vaccination with Exomyc complex, it is necessary to separately obtain the antigen included in the complex. The strategy used in this work is illustrated in FIG. 12. Initially, the coding sequences for the polyprotein P36-MHP271, P46-P97R1 R2 and HSP-NrdF were separated from pMRK-Exomyc plasmids, using cuts with the enzymes BgIII-Pstl, BamHI-EcoRI and Xhol-HindIII, respectively, to be cloned into the respective sites of the pMRK vector.

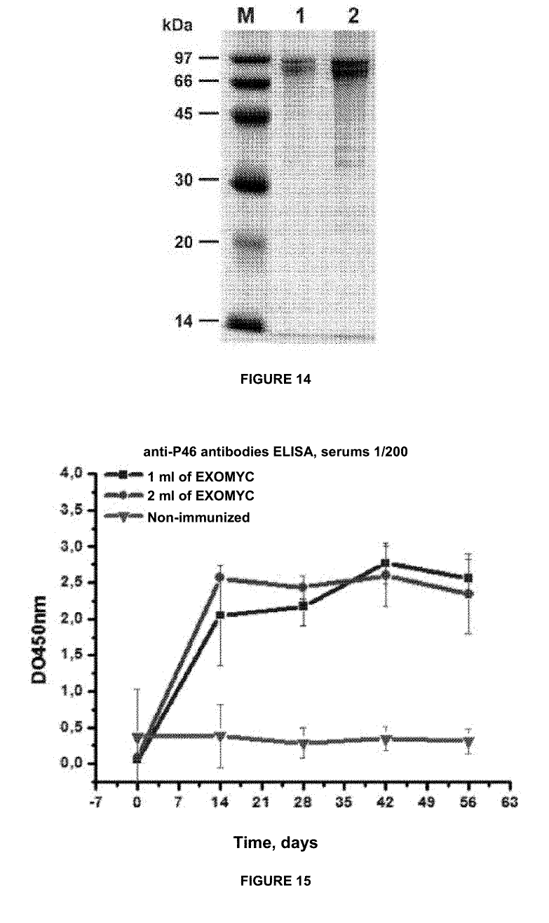

Then, the gene of each antigen was amplified by PCR using a specific primer, and a sequencing primer T7 and T7rev. Thus, 6 plasmides were obtained (pMRK-P36, pMRK-MHP271, pMRK-P46, pMRK-P97R1 R2, pMRK-HSP and pMRK-NrdF) expressing the antigen with fusion of 6× His. Of these constructions only pMRK-P46 and the pMRK-HSP express the 6× His-P46 and 6× His-HSP proteins at a high level and in soluble form. Other antigens were cloned into the pET-SUMO vector, to its fusion with SUMO protein, obtaining the pET-SUMO-P36, pET-SUMO-MHP271, pET-SUMO-P97R1 R2, pET-SUMO-NrdF plasmids. Except the construction pET-SUMO-P36, all expressed in high level and in soluble form, thus obtaining the SUMO-MHP271, SUMO-P97R1 R2 e SUMO-NrdF proteins. FIG. 14 shows the result of purification of antigens separately. All proteins except P36 were able to be purified with high degree of purity by affinity chromatography on immobilized metal (IMAC) and gel-filtration. Only the SUMO-MHP271 protein proved to be unstable during storage to 4° C., passing to show a smaller protein (in FIG. 12A with asterisk indicates the migration of the main band after purification).

### Example 3

### Expression of EXOMYC Complex in E. coli Cells

The Escherichia coli BL21 strain (DE3) transformed with the pMRK-EXOMYC plasmid was grown in 5 ml of LB Kanamycin medium (25 ug/ml) and incubated with the material of a seed bank cryotube (selected product clone, consistent in Escherichia coli cells transformed with the pMRK-EXOMYC plasmid, frozen at −80° C. in the presence of glycerol). The culture was incubated at 37° C., at 200 rpm, by 16 hours.

One milliliter of culture was inoculated into 25 ml of LB Kanamycin medium (25 ug/ml) (A) and 25 ml of LB Kanamycin medium (25 ug/ml)+Lactose 0,8%, Glycerol 0.5% and MgSO4 1 mM (self-induction). The cultures were incubated at 37° C., at 200 rpm.

Culture A.—After the culture reaches DO_{600 nm} of 0.5, it was added ITPG in the final concentration of 1 mM and it was incubated additional 4 hours at 28° C.

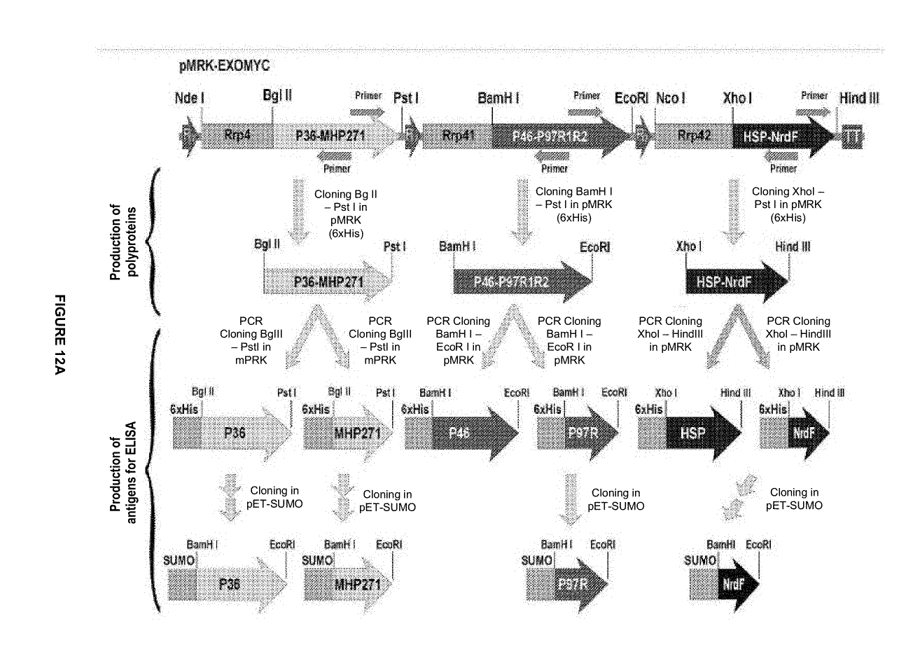

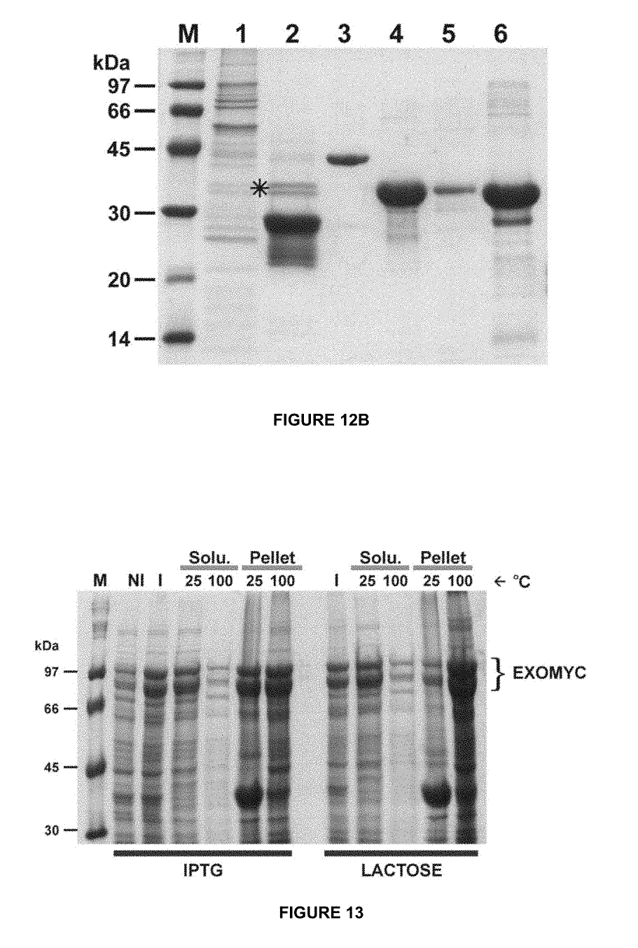

Culture B.—After 4 hours at 37° C., it was incubated at 28° C. for additional 6 hours. Aliquots of the cultures were analyzed in gel of SDS-PAGE (FIG. 13) before (NI) and after induction (I).

Cells were collected by centrifugation at 6000 rpm for 30 minutes and re-suspended in lysis buffer (50 mM Tris-HCI, pH 8.0; 0.1% Triton X-100; 0.25% Sarcozyl) and lysed in a high pressure homogenizer (800 bar-80×10³ kPa).

Aliquots of the lysates were incubated at 25 and 100° C. for 30 minutes, cooled on ice and centrifuged for 15 minutes 12000 rpm. Both the supernatant and the pellet were analyzed by SDS-PAGE (FIG. 13). For this experiment, it was determined that Exomic complex is expressed both in soluble form and in inclusion bodies, and the proportion of soluble recombinant protein is greater in the induction with lactose. The treatment with temperature of 100° C. showed that the immunogenic proteins are fused to the proteins of the P. abyssi exosome make complex which is not thermostable, the difference of the exosome with or without fusions and smaller confusion.

### Example 4

### Production of EXOMYC Complex in E. coli Cells

The strain of Escherichia coli BL21 (DE3) transformed with the pMRK-EXOMYC plasmid was grown in 200 ml of LB Kanamycin medium (25 ug/ml) and incubated with the material of a seed band cryotube. The culture was incubated at 37° C., in 200 rpm, for 16 hours.

The 200 ml of culture were inoculated in 5 liters of MCHD medium (Complex Medium of High Density) in fermenter of 15 liters. The fermentation was performed in steady-fed batch, at 37° C., 30% of dissolved oxygen having glycerol as carbon source. In the DO_{600 nm}=15, feeding of glycerol was replaced by lactose (induction) and the fermentation continued for 12 hours at 28° C.

Cells were collected by centrifugation at 6000 rpm for 30 minutes and re-suspended in lysis buffer (50 mM Tris-HCI, pH 8.0; 0.1% Triton X-100; 0,25% Sarcozyl) and lysed in a high pressure homogenizer.

The lysed and homogenized cells were subjected to filtration through a membrane of 0.2 μm (millipore “end-filtration” system) at room temperature.

It was added MgCl₂ for final concentration of 5 mM and Benzonase 90 u/ml (final concentration), and the mixture was incubated at room temperature for 6 hours.

The material treated with Benzonase was concentrated and diafiltrated in the tangential filtration system using a hollow fiber membrane of 500 kDa of MWCO, in the buffer 20 mM Tris-HCl, pH 8.0; 100 mM NaCl and 0,1 mM EDTA. The protein retained is sterilized by microfiltration on membrane of 0.2 μm and stored at 4° C. FIG. 14 shows the result of co-filtration of polyproteins comprising the Exomyc complex.

### Example 5

### Formulation with EXOMYC Complex for Animals

The complex of Exomyc polyproteins was formulated in double emulsion oil (Montanide ISA 206) with saponin as adjuvant, with the components shown in Table 7.

TABLE 7
Composition of Exomyc vaccine, for 1 ml.

| Component | Amount | |
|---|---|---|
| EXOMYC | 200 | μg |
| Saponine | 1 | mg |
| Timerosal | 0.01 in aqueous phase | |
| Moontanide ISA 206 | 0.5 | ml |

The animals (swines) were grouped in 8. The first group A received a single dose of 1 ml of vaccine (200 μg of antigen), the second group B received a single dose of 2 ml of vaccine (400 μg of antigen) and the third group C was not vaccinated. The followed chronogram was as follows:

| | |
|---|---|
| Day 0 | Serum collection and first and single dose |
| Day 14 | Serum collection |
| Day 28 | Serum collection |
| Day 42 | Serum collection |
| Day 56 | Serum collection |
| Day 140 | Sacrifice |

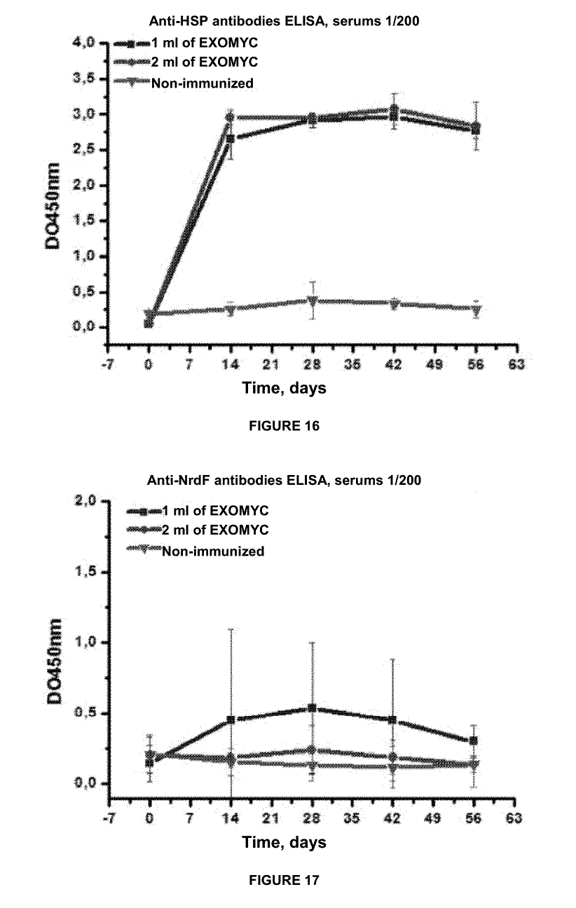

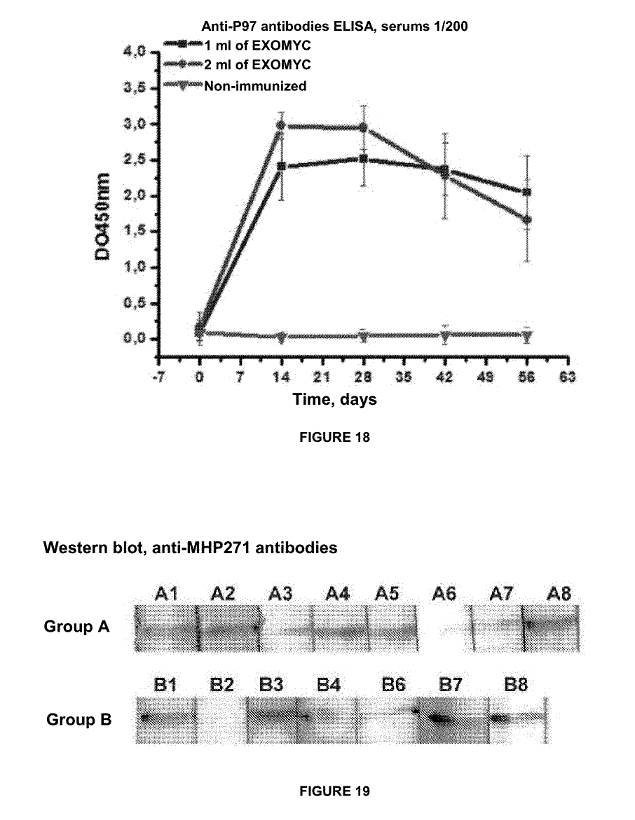

The results of the analyzes by ELISA of the sera to determine antibodies against antigens included in the Exomyc complex, performed with proteins purified in Example 2, are shown in FIG. 15 (P46), FIG. 16 (HSP), FIG. 17 (NrdF) and FIG. 18 (P97R1 R2). The response against the MHP217 protein was characterized by Western blot (FIG. 19).

These results confirm the presence of the tested proteins in Exomyc complex, that is, it is truly possible to co-express, in soluble form, three polyprotein of large size and the co-purify in a single complex (Exomyc). In addition, the immunization with this complex can induce responses against each of the antigens present on it.

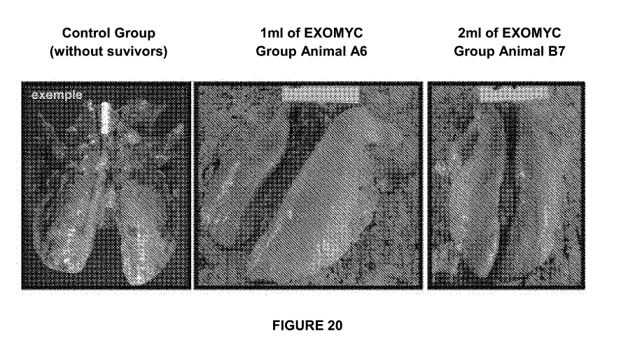

During the experiment, the animals that have not been vaccinated with any vaccine died from diseases associated with PES (Enzzotic Pneumonia of Swine) and CRS (Swine Respiratory Complex). After sacrifice of the animals immunized with 1 or 2 ml of Exomyc vaccine, few lesions were found in the lungs and liver, and some animals in Group B showed no injury (FIG. 20). Thus it was proved the effectiveness of the vaccine containing the Exomyc complex.

We claim:

1. A complex of immunogenic polyproteins of M. hyopneumoniae comprising immunogenic and stable segments derived from P36, P46, HSP (P42), NrdF, P97, MHP271 and P216 antigens of M. hyopneumoniae, fused to segments of P. abyssi exosome, wherein the major segments of amino acids derived from M. hyopneumoniae antigens are directly linked to proteins of said P. abyssi exosome, while smaller segments are at the C-terminal extreme of the fusions.

2. A complex of immunogenic polyproteins according to claim 1, wherein the proteins of P. abyssi exosome selected for fusion with the immunogenic and stable segments derived from P36, P46, HSP (P42), NrdF, P97, MHP271 and P216 antigens of M. hyopneumoniae are Rrp4, Rrp41 and Rrp42, wherein the stability of the complex is reached by interaction of said proteins, thus maintaining all antigens in a single proteic complex.

3. A complex of immunogenic polyproteins of M. hyopneumoniae according to claim 1, comprising an amino acids sequence selected from:
(a) SEQ ID NO:26, SEQ ID NO:27 and SEQ ID NO:28;
(b) conservatively modified variants having at least 85% of identity in relation to amino acids sequences of the sequences SEQ ID NO:26, SEQ ID NO:27 and SEQ ID NO:28, and having substantially the same immunogenic activity thereof;
(c) fragments of sequences SEQ ID NO:26, SEQ ID NO:27 and SEQ ID NO:28, said fragments comprising immunogenic and stable regions of P36, P46, HSP (P42), NrdF, P97, MHP271 and P216 antigens; and
(d) a fusion protein encoded by nucleotide sequence of SEQ ID NO: 25, a conservatively modified variant of said fusion protein or a fragment of said fusion protein having the same immunogenic activity.

4. A complex of immunogenic polyproteins according to claim 3, comprising the amino acids sequences of SEQ ID NO:26, SEQ ID NO:27 and SEQ ID NO:28.

5. A complex of immunogenic polyproteins according to claim 3, comprising a fusion protein encoded by nucleotide sequence of SEQ ID NO:25.

6. A gene encoding a complex of immunogenic polyproteins of M. hyopneumoniae comprising nucleotide sequences encoding immunogenic and stable segments derived from P36, P46, HSP (P42), NrdF, P97, MHP271 and P216 antigens of M. hyopneumoniae, and encoding nucleotide sequences of segments of P. abyssi exosome, forming a fusion protein of said immunogenic and stable segments with said segments of P. abyssi exosome so that the encoding nucleotide sequences of greater segments of amino acid derived from M. hyopneumoniae antigens are directly linked to nucleotide sequences encoding the proteins of said P. abyssi exosome, while the nucleotide sequences encoding said minor segments of amino acids derived from M. hyopneumoniae antigens are in the C-terminal extreme of the fusion protein.

7. A gene according to claim 6, wherein the encoding nucleotide sequences of proteins of P. abyssi exosome selected for fusion with the immunogenic and stable segments specific of P36, P46, HSP (P42), NrdF, P97, MHP271 and P216 antigens of M. hyopneumoniae are the encoding nucleotide sequences of Rrp4, Rrp41 and Rrp42 proteins of P. abyssi.

8. A gene encoding a complex of immunogenic polyproteins of M. hyopneumoniae according to claim 6, comprising a nucleotide sequence selected from group consisting of:
(a) SEQ ID NO:25; and
(b) a nucleotide sequence having at least 75% of identity related to SEQ ID NO:25.

9. A gene according to claim 8, wherein said nucleotide sequence encodes the polyproteins comprising the sequences SEQ ID NO:26 SEQ ID NO:27 and SEQ ID NO:28.

10. A process for obtaining a complex of immunogenic polyproteins, comprising the steps of:
(i) preparation of an expression system comprising:
(a) a gene encoding a complex of polyproteins of M. hyopneumoniae comprising nucleotide sequences encoding immunogenic and stable segments derived from P36, P46, HSP (P42), NrdF, P97, MHP271 and P216 antigens of M. hyopneumoniae and encoding nucleotide sequences of segments of P. abyssi exosome, forming a fusion protein of said immunogenic and stable segments with said segments of P. abyssi exosome so that the encoding nucleotide sequences of greater segments of amino acid derived from M. hyopneumoniae antigens are directly linked to nucleotide sequences encoding the proteins of said P. abyssi exosome, while the nucleotide sequences encoding said minor segments of amino acids derived from M. hyopneumoniae antigens are in the C-terminal extreme of the fusion protein;
(b) an expression system, optionally selected from Escherichia coli strain BL21 (DE3) or Rosetta (DE3), and
(c) an expression vector containing the phage promoter T7;
(ii) innoculation of host cells with the expression system prepared in step (i);
(iii) lysing the cells to obtain said complex of immunogenic polyproteins of M. hyopneumoniae; and
(iv) purification of said complex obtained in step (iii).

11. The process according to claim 10, wherein the encoding gene of step (i) (a) is formed by DNA amplification of primer sequences: SEQ ID NO:29, SEQ ID NO:30, SEQ ID NO: 31 , SEQ ID NO:32, SEQ ID NO:33, SEQ ID NO:34, SEQ ID NO:35 and SEQ ID NO:36.

12. The process according to claim 10, wherein said expression vector containing the phage promoter T7 of step (i) (c) is selected from the group consisting of plasmid of pET series and the pMRK plasmid.

13. The process according to claim 12, wherein said plasmid of pET series is pRSET.

14. The process according to claim 12, wherein said plasmid is pMRK.

15. An antigenic composition, comprising:
(a) complex of immunogenic polyproteins of M. hyopneumoniae of claim 1;
(b) optionally, an adjuvant; and
(c) a pharmaceutically acceptable vehicle.

16. The antigenic composition according to claim 15, wherein said adjuvant is selected from group consisting of: incomplete Freund's adjuvant, the complete Freund's adjuvant, polydisperse acetylated mannan β-(1,4) linked, adjuvants of copolymer of polyoxiethylene-polyoxipropylene, modified lipid adjuvants, adjuvants derived from saponine, dextran sulfate, aluminum hydroxide and aluminum phosphate.

17. The antigenic composition according to claim 15, wherein said pharmaceutically acceptable vehicle includes preservatives, diluents, emulsifiers, stabilizers and other pharmaceutically acceptable ingredients.

18. A method of treatment of infection caused by Mycoplasma hyopneumoniae, the method comprising administration of the complex of immunogenic polyproteins of M. hyopneumoniae as defined in claim 1 to a patient in need of treatment.

19. A method of prophylaxis of infections caused by Mycoplasma hyopneumoniae, the method comprising administration of the complex of immunogenic polyproteins of M. hyopneumoniae as defined in claim 1 to a patient in need of prophylaxis.

20. The process of claim 10, wherein the gene encoding a of complex of polyproteins comprises the sequences SEQ ID NO:26, SEQ ID NO:27 and SEQ ID NO:28.
